SEQUENCE LISTING (23 sequences)

SEQ ID NO: 1 - DNA, 32 bp, Artificial Sequence
  Description of Artificial Sequence Synthetic primer
ggaacacgcacgccatgggtaaacccattgtg

SEQ ID NO: 2 - DNA, 30 bp, Artificial Sequence
  Description of Artificial Sequence Synthetic primer
gatgtaagtaattactgcaggattacttaa

SEQ ID NO: 3 - DNA, 31 bp, Artificial Sequence
  Description of Artificial Sequence Synthetic primer
gattccttgcatcatatgatgtacggagttt

SEQ ID NO: 4 - DNA, 35 bp, Artificial Sequence
  Description of Artificial Sequence Synthetic primer
tccgattccgatctagattaatgatgagcccattc

SEQ ID NO: 5 - DNA, 33 bp, Artificial Sequence
  Description of Artificial Sequence Synthetic primer
caggaggtgccatatgattttttccgtcgacaa

SEQ ID NO: 6 - DNA, 34 bp, Artificial Sequence
  Description of Artificial Sequence Synthetic primer
ccatagtgcctctagattatcccagcaaacgtga

SEQ ID NO: 7 - DNA, 29 bp, Artificial Sequence
  Description of Artificial Sequence Synthetic primer
gccgaggagtcatatgaacgtttttaatc

SEQ ID NO: 8 - DNA, 30 bp, Artificial Sequence
  Description of Artificial Sequence Synthetic primer
gcgggttctagattaatccaccaataattc

SEQ ID NO: 9 - DNA, 35 bp, Artificial Sequence
  Description of Artificial Sequence Synthetic primer
accaaatctagaactcttattttgcttcacgtttg

SEQ ID NO: 10 - DNA, 31 bp, Artificial Sequence
  Description of Artificial Sequence Synthetic primer
ggctagggcccaataatgtatagacgatctg

SEQ ID NO: 11 - DNA, 28 bp, Artificial Sequence
  Description of Artificial Sequence Synthetic primer
cggcctcgagcattatttcatgtttctt

SEQ ID NO: 12 - DNA, 33 bp, Artificial Sequence
  Description of Artificial Sequence Synthetic primer
ccggactagtaagatgttgaaatcaactgctac

SEQ ID NO: 13 - DNA, 35 bp, Artificial Sequence
  Description of Artificial Sequence Synthetic primer
gcggcttaattaatcaatgacctgaccatttgatg

SEQ ID NO: 14 - DNA, 1691 bp, Saccharomyces cerevisiae
acgcatttttttcttttgcattttatcttttcccttaatatgtcatgagaataaaatattaaggatataccgtaaatatacaaattcgtatcgaatatttgatattgtttcctaacttttctgtgttaaaaataaatatgcaaaaacatactaaccataaaaataaataaaatacaagatataaataaataatccagcaagcaaagacattgcggtccatgccataattgttttcttccaactattagatttcagtgtgcattatttcatgtttcttttcttcaaaacgttctcaataaatgcttcaccagccttatatgacgatcttactaggggtccagatgcacaatacaagaatcccatctctaaagctctctctttccagtagtcaaacttttcgggtttcacatattctacgactttcatatgtctcttggttggcctcatatattgaccaaatgtaacaacatcacattgaatattgcgcaaatccttcaaagtttgagtaatttgctcatcagtttctcctagacccagcattattgatgttttagtaatcagtgacggaaccgtagcttttgccctttctaaaacactcaaagactgtctataagtagctcttctgtctctgacatgtggtgttagtgattcaactgtttccaaattatgtgcgtaaacatcaagcccacattgtgccataatgtccaccatcttcaaatcacctctgaaatcaccagaaagagtctctacaagagtatttggtgccttctgtttgattttgcgaaccgtctcggccaggtgattagcaccaccatcgactaaatcgtccctatcaacggtagttaaaacaacataacccaacccccatcttttgatagcttcggcagtattttcgggctccattgggtccggcttactaggcgttctattggtcttcacagaacaaaacctacatccacgagtgcaagtatcaccaagcagcataattgttgccgttgccttagatttatctttgcctccccaacattcaccaatattgggacatcttgcctcctcacaaacagtactcaggcctaattcttttacgtcgcctttcaatttatggtaattggtacccttaggaataggaaccttaagccaacgaggtagttttttagcttcctctgtattttgccttgccttctccagagggtccaagatcattttcgaagctttacctgaaacaaaatctgcaaaagatggacccaaattaagagcatctttgaactctgtaattctttttctgttccccttgcgtctttttcccgaagtccctgttaactgactggttgcattttcaacctcagttgaatttccaactggtaccctaacgctagcgttatctgaatcagtattcaatgcattggatcttattggccgagttgcgctcgttccacacctaatagtggacgaaatccatcttgtatttctcccaacaaatagtactccaacagatcgtctatacattattgtggcttaacagttctccttaaaatccacataactgacaaaactggatacttaatgcccatcgagtcatatacatatatttgattactacaagtgcttttcaaataactgcttcgagaaaagcgtccgggtaataacaactattgaaaaagcatggcttcgcattaataggagccaaaaat

SEQ ID NO: 15 - protein, 414 aa, Saccharomyces cerevisiae
MYRRSVGVLFVGRNTRWISSTIRCGTSATRPIRSNALNTDSDNASVRVPVGNSTEVENATSQLTGTSGKRRKGNRKRITEFKDALNLGPSFADFVSGKASKMILDPLEKARQNTEEAKKLPRWLKVPIPKGTNYHKLKGDVKELGLSTVCEEARCPNIGECWGGKDKSKATATIMLLGDTCTRGCRFCSVKTNRTPSKPDPMEPENTAEAIKRWGLGYVVLTTVDRDDLVDGGANHLAETVRKIKQKAPNTLVETLSGDFRGDLKMVDIMAQCGLDVYAHNLETVESLTPHVRDRRATYRQSLSVLERAKATVPSLITKTSIMLGLGETDEQITQTLKDLRNIQCDVVTFGQYMRPTKRHMKVVEYVKPEKFDYWKERALEMGFLYCASGPLVRSSYKAGEAFIENVLKKRNMK

SEQ ID NO: 16 - DNA, 1494 bp, Saccharomyces cerevisiae
atgttgaaatcaactgctacaagatcgataacaagattatctcaagtttacaacgttccagcggccacatatagggcttgtttggtaagcaggagattctattcccctcctgcagcaggcgtgaagttagacgacaacttctctctggaaacgcataccgatattcaggctgctgcaaaggcacaggctagtgcccgtgcgagtgcatccggtaccaccccagatgctgtagtagcttctggtagcactgcaatgagccatgcttatcaagaaaacacaggttttggtactcgtcccatatatcttgacatgcaagccactacaccaacagaccctagggttttggatacgatgttgaagttttatacgggactttatggtaatcctcattccaacactcactcttacggttgggaaacaaatactgctgtggaaaatgctagagctcacgtagcaaagatgatcaatgccgaccccaaggaaataatattcacttcgggagcgaccgaatctaataatatggttcttaagggtgtcccaagattttataagaagactaagaaacacatcatcaccactagaacggaacacaagtgtgtcttggaagccgcacgggccatgatgaaggagggatttgaagtcactttcctaaatgtggacgatcaaggtcttatcgatttgaaggaattggaagatgccattagaccagatacctgtctcgtctctgtgatggctgtcaataatgaaatcggtgtcattcaacctattaaagaaattggtgcaatttgtagaaagaataagatctactttcatactgacgccgcacaagcctatggtaagattcacattgatgtcaatgaaatgaacattgatttactatcaatttcttctcacaagatttacggtccaaagggaataggtgccatctatgtaagaaggagaccaagagttagattagaacctttactatccggtggtggccaagagagaggattgagatctggtactttggcccccccattggtagcgggatttggtgaagctgcgagattgatgaagaaagaatttgacaacgaccaagctcacatcaaaagactatccgataaattagtcaaaggtctattatccgctgaacataccacgttgaacggatctccagatcatcgttatccagggtgtgttaacgtttctttcgcctacgtggaaggagaatctttattgatggcactaagggatatcgcattatcctcgggttcagcctgtacatctgcttccctagaaccttcttatgttttacatgcgctgggtaaggatgatgcattagcccattcttccatcagatttggtattggtagatttagtactgaagaggaggtcgactacgtcgttaaggccgtttctgacagagtaaaattcttgagggaactttcaccattatgggaaatggttcaagaaggtattgacttaaactccatcaaatggtcaggtcattga

SEQ ID NO: 17 - protein, 497 aa, Saccharomyces cerevisiae
MLKSTATRSITRLSQVYNVPAATYRACLVSRRFYSPPAAGVKLDDNFSLETHTDIQAAAKAQASARASASGTTPDAVVASGSTAMSHAYQENTGFGTRPIYLDMQATTPTDPRVLDTMLKFYTGLYGNPHSNTHSYGWETNTAVENARAHVAKMINADPKEIIFTSGATESNNMVLKGVPRFYKKTKKHIITTRTEHKCVLEAARAMMKEGFEVTFLNVDDQGLIDLKELEDAIRPDTCLVSVMAVNNEIGVIQPIKEIGAICRKNKIYFHTDAAQAYGKIHIDVNEMNIDLLSISSHKIYGPKGIGAIYVRRRPRVRLEPLLSGGGQERGLRSGTLAPPLVAGFGEAARLMKKEFDNDQAHIKRLSDKLVKGLLSAEHTTLNGSPDHRYPGCVNVSFAYVEGESLLMALRDIALSSGSACTSASLEPSYVLHALGKDDALAHSSIRFGIGRFSTEEEVDYVVKAVSDRVKFLRELSPLWEMVQEGIDLNSIKWSGH

SEQ ID NO: 18 - DNA, 60 bp, Artificial Sequence
  Description of Artificial Sequence Synthetic primer
aacgctttccttcgtaattcgcaactggaacacgcacgctgtgtaggctggagctgcttc

SEQ ID NO: 19 - DNA, 60 bp, Artificial Sequence
  Description of Artificial Sequence Synthetic primer
cgggttttttatcagacagatgtaagtaattattacaggacatatgaatatcctccttag

SEQ ID NO: 20 - DNA, 22 bp, Artificial Sequence
  Description of Artificial Sequence Synthetic primer
ggtatctatgggtgagattagt

SEQ ID NO: 21 - DNA, 25 bp, Artificial Sequence
  Description of Artificial Sequence Synthetic primer
gtccttaaatgaggagcaaatagat

SEQ ID NO: 22 - DNA, 20 bp, Artificial Sequence
  Description of Artificial Sequence Synthetic primer
aggtcgtgtactgtcagtca

SEQ ID NO: 23 - DNA, 20 bp, Artificial Sequence
  Description of Artificial Sequence Synthetic primer
acgtggtgaactgccagtga

(19) United States
(12) Patent Application Publication
(10) Pub. No.: US 2004/0235123 A1
(43) Pub. Date: Nov. 25, 2004

(54) Production of alpha-lipoic acid

(76) Inventors: Hans H Liao, Eden Prairie, MN (US); Sara C McFarlan, St Paul, MN (US)

Correspondence Address: Scott R Pribnow, Cargill Inc, Law Department, P O Box 5624, Minneapolis, MN, 55440-5624, US

(21) Appl. No.: 10/475,311

(22) PCT Filed: Apr. 19, 2002
(86) PCT No.: PCT/US02/12458

Related U.S. Application Data
(60) Provisional application No. 60/285,276, filed on Apr. 20, 2001.

(51) Int. Cl. C12P 17/00; C12N 1/21

(52) U.S. Cl. 435/117; 435/252.33

(57) ABSTRACT

Engineered microorganisms that produce at least 200 μg alpha-lipoic acid (ALA) per g dry cell weight and engineered microorganisms that secrete at least a 2-fold greater amount of ALA than an amount of ALA found intracellularly, are described.

### CROSS REFERENCE TO RELATED APPLICATION

This claims the benefit of U.S. Provisional Application No. 60/285,276 filed Apr. 20, 2001.

### FIELD

The disclosure relates to methods and materials and methods for producing α-lipoic acid (ALA), as well as nucleic acids, polypeptides, and engineered microorganisms that can be used for producing ALA.

### BACKGROUND

Alpha-lipoic acid (ALA, 6,8-thioctic acid, 1,2-dithiolane-3-pentanoic acid) is found in low levels (<35 μg ALA per g dry cell weight) in a wide variety of microorganisms, as well as in plants and animals. It is a cofactor for the α-keto acid dehydrogenase enzyme complexes (pyruvate dehydrogenase and α-ketoglutarate dehydrogenase) and the glycine cleavage system (Self et al., Proc. Natl. Acad. Sci. USA 97:12481-6, 2000). Both oxidized and reduced forms of ALA have antioxidant abilities against oxidative stress-induced processes, and are potent scavengers of a variety of reactive oxygen species. The reduced form can regenerate other natural antioxidants, such as glutathione, vitamin C, ubiquinone, thioredoxin, and Vitamin E. ALA has been prescribed for nearly thirty years in Europe to treat diabetic polyneuropathy, regulate blood sugar, and to prevent diabetic retinopathy and cardiopathy.

Commercially available ALA is synthesized chemically and sold as a racemic mixture of the R and S forms. The R form is found in nature and is the active form found in enzymes, although both enantiomers have antioxidant properties.

### SUMMARY

Disclosed are methods and materials for producing ALA in an engineered microorganism. ALA produced by the disclosed microorganisms can be used directly, used as a nutritional supplement, and can be formulated for use in pharmaceutical compositions for treatment, for example alleviating or relieving a symptom of a disorder in a subject being treated, of a variety of conditions, including, for example, side effects from diabetes, glaucoma, and liver disorders. Isolated nucleic acids that can be used to engineer microorganisms which have the ability to produce large quantities of non-racemic, enantiomerically pure ALA are disclosed.

Engineered microorganisms that produce at least 200 μg ALA per g dry cell weight of the microorganism, such as at least 1000 μg ALA per g dry cell weight, and compositions containing such engineered microorganisms, are disclosed. In one example, engineered microorganisms include an exogenous nucleic acid encoding a lipoic acid synthase polypeptide, and in some examples, further includes at least one polypeptide involved in the assembly or regeneration of the Fe—S clusters of the lipoic acid synthase polypeptide. Examples of polypeptides involved in the assembly or regeneration of the Fe—S clusters of the lipoic acid synthase polypeptide include the nitrogen fixation U polypeptide (NifU, also referred to herein as an iron-sulfur cluster polypeptide U, IscU) and the nitrogen fixation S polypeptide (NifS, also referred to herein as an iron-sulfur cluster polypeptide S, IscS), such as cysteine sulfinic desulfinase (Csd). In a particular example, the microorganism includes an exogenous lipoic acid synthase polypeptide, an exogenous IscS polypeptide, and/or an exogenous IscU polypeptide. The microorganism can be a prokaryote, such as a member of the Escherichia genus, for example E. coli. In one example, the microorganism lacks an endogenous transcriptional regulator IscR ORF.

Also disclosed are engineered microorganisms that secrete at least a 2-fold greater amount of ALA than an amount of intracellular ALA content, and compositions containing such engineered microorganisms, are disclosed. In one example, the microorganism secretes at least a 5-fold, such as at least a 10-fold, such as at least a 20-fold greater amount of ALA than an amount of intracellular ALA. Alternatively or in addition, the microorganism secretes at least 8.7 μg ALA per g dry cell weight and contains less than 1.5 μg ALA per g dry cell weight intracellularly. The microorganism can be a eukaryote, such as a member of the Saccharomyces genus, for example S. cerevisiae. In one example, engineered microorganisms include an exogenous nucleic acid encoding a lipoic acid synthase polypeptide, and in some examples, further includes at least one polypeptide involved in the assembly or regeneration of the Fe—S clusters of the lipoic acid synthase polypeptide, such as an Nfs1 polypeptide. In a particular example, the microorganism includes an exogenous lipoic acid synthase polypeptide and/or an exogenous Nfs1 polypeptide.

Also disclosed is an isolated nucleic acid that includes a promoter operably linked to a nucleic acid encoding a lipoic acid synthase and a promoter operably linked to a nucleic acid encoding one or more polypeptides involved in the assembly or regeneration of the Fe—S clusters of the lipoic acid synthase, such as an Nfs1 polypeptide, an IscS polypeptide, for example Csd, and/or an IscU polypeptide. Such nucleic acids can be expressed in a microorganism to permit production of ALA by the microorganism. The nucleic acids disclosed herein can be part of a vector. Cells including the disclosed nucleic acids and vectors are also comprehended by this disclosure.

Methods of making ALA from the disclosed engineered microorganisms are disclosed. In one example, the method includes extracting ALA from an engineered microorganism which produces at least 200 μg ALA per g dry cell weight. In another example, the method includes purifying or concentrating ALA secreted by an engineered microorganism which secretes at least a 2-fold greater amount of ALA than an amount of intracellular ALA. The ALA produced by the disclosed engineered microorganisms can be used alone, or in combination with one or more pharmaceutically acceptable carriers, and/or in combination with other therapeutic compounds, for example to produce nutraceuticals and pharmaceuticals. Pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the nucleic acids herein disclosed.

Methods of making ALA by expressing the disclosed nucleic acids in a microorganism are also disclosed. For example, an exogenous nucleic acid encoding a lipoic acid synthase polypeptide, alone or in combination with an exogenous nucleic acid encoding at least one polypeptide that assembles or regenerates an Fe—S cluster of the lipoic acid synthase polypeptide, can be expressed in a microorganism. The microorganism is cultured under conditions which permit the microorganism to produce ALA. In some examples, the microorganism produces at least 200 μg ALA per g dry cell weight. In other examples, the microorganism secretes at least a 2-fold greater amount of ALA than an amount of ALA found intracellularly.

Compositions, such as nutraceuticals and pharmaceuticals, which include ALA produced by the disclosed engineered microorganisms are disclosed. In one example, such compositions include one or more pharmaceutically acceptable carriers, and/or one or more additional therapeutic compounds.

### DESCRIPTION OF THE FIGURES

FIG. 1 is a schematic of the route for production of alpha-lipoic acid (ALA).

FIG. 2 is an ALA standard curve.

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NOS: 1 and 2 are primers used to amplify an E. coli lipA.

SEQ ID NOS: 3 and 4 are primers used to amplify an E. coli nifS-I.

SEQ ID NOS: 5 and 6 are primers used to amplify an E. coli nifS-II.

SEQ ID NOS: 7 and 8 are primers used to amplify an E. coli csd.

SEQ ID NO: 9 is a primer used to amplify an E. coli nifS-I/nifU gene cluster.

SEQ ID NOS: 10 and 11 are primers used to amplify an S. cerevisiae lip5.

SEQ ID NOS: 12 and 13 are primers used to amplify an S. cerevisiae nfs1.

SEQ ID NO: 14 is an S. cerevisiae lip5 nucleotide cDNA sequence (GenBank Accession No. 1420466; ORF YOR196c; ChrXV: coordinates 716836 to 715592).

SEQ ID NO: 15 is an S. cerevisiae Lip5 amino acid sequence (GenBank Accession No. CAA99409.1; GI:1420467).

SEQ ID NO: 16 is an S. cerevisiae nfs1 nucleotide cDNA sequence (GenBank Accession No. 10383748; ORF YCL017C; ChrIII: coordinates 94269 to 92776).

SEQ ID NO: 17 is an S. cerevisiae Nfs1 amino acid sequence (GenBank Accession No. NP_{—}009912.2; GI:10383773).

SEQ ID NOS: 18 and 19 are primers used to amplify a chloramphenicol acetyltranferase gene on pKD3.

SEQ ID NOS: 20 and 21 are primers used to confirm integration of a CAT gene into a lipA locus.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a protein” includes one or a plurality of such proteins, and reference to “comprising the microorganism” includes reference to one or more microorganisms and equivalents thereof known to those skilled in the art, and so forth.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features and advantages of the disclosure will be apparent from the following detailed description, and from the claims.

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Comprises: A term that means “including.” For example, “comprising A or B” means including A or B, or both A and B, unless clearly indicated otherwise.

Conservative substitution: One or more amino acid substitutions (for example at least 2, 5 or 10 residues) for amino acid residues having similar biochemical properties. Typically, conservative substitutions have little to no impact on the activity of a resulting polypeptide. For example, ideally, a lipoate synthase peptide including one or more conservative substitutions retains lipoate synthase activity. A polypeptide can be produced to contain one or more conservative substitutions by manipulating the nucleotide sequence that encodes that polypeptide using, for example, standard procedures such as site-directed mutagenesis or PCR.

Similar amino acids are those that are similar in size and/or charge properties. Families of amino acids with similar side chains are known. These families include amino acids with basic side chains (e.g., lysine, arginine, or histidine), acidic side chains (e.g., aspartic acid or glutamic acid), uncharged polar side chains (e.g., asparagine, glutamine, serine, threonine, tyrosine, or cysteine), nonpolar side chains (e.g., alanine, valine, glycine, leucine, isoleucine, proline, phenylalanine, methionine, or tryptophan), branched side chains (e.g., threonine, valine, or isoleucine), and aromatic side chains (e.g., tyrosine, phenylalanine, tryptophan, or histidine).

Other examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found in, among other locations in, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

In one example, such variants can be readily selected for additional testing by performing an assay. For example, variants can tested for an ability to synthesize ALA from octanoic acid esters (i.e. whether a lipoate synthase variant retains lipoate synthase activity) or for an ability to assemble or regenerate Fe—S clusters of lipoate synthase (i.e. whether an IscS, Nfs1 and/or IscU variant retains IscS, Nfs1 and/or IscU activity, respectively) using the methods described in EXAMPLES 1-5.

Deletion: The removal of a sequence of a nucleic acid, for example DNA, the regions on either side being joined together.

DNA: Deoxyribonucleic acid. DNA is a long chain polymer which comprises the genetic material of most living organisms (some viruses have genes comprising ribonucleic acid, RNA). The repeating units in DNA polymers are four different nucleotides, each of which comprises one of the four bases, adenine, guanine, cytosine and thymine bound to a deoxyribose sugar to which a phosphate group is attached (however one skilled in the art will understand that variant nucleotides can also be used). Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Exogenous: The term “exogenous” as used herein with reference to a nucleic acid and a particular cell or microorganism refers to any nucleic acid that does not originate from that particular cell/microorganism. Thus, a non-naturally-occurring nucleic acid is considered to be exogenous to a cell/microorganism once introduced into the cell/microorganism. A nucleic acid that is naturally-occurring also can be exogenous to a particular cell/microorganism. For example, an operon or other nucleic acid sequence isolated from a bacteria is an exogenous nucleic acid with respect to a second bacteria once that operon or other nucleic acid sequence is introduced into the second bacteria.

In one example, a non-naturally-occurring nucleic acid contains nucleic acid sequences or portions thereof that are found in nature, but the nucleic acid as a whole does not exist in nature. For example, a nucleic acid molecule containing a genomic DNA sequence within an expression vector is a non-naturally-occurring nucleic acid, and this is exogenous to a cell/microorganism once introduced into the cell/microorganism, since that nucleic acid molecule as a whole (genomic DNA plus vector DNA) does not exist in nature. In another example, a nucleic acid which includes a promoter sequence and a sequence encoding a polypeptide in an arrangement not found in nature is a non-naturally occurring nucleic acid.

Functional deletion: A mutation, partial or complete deletion, insertion, or other variation made to a gene sequence which inhibits production of the gene product, and/or renders the gene product non-functional.

Hybridization: A method of testing for complementarity of a nucleotide sequence between two or more nucleic acid molecules, based on the ability of complementary single-stranded DNA and/or RNA to form a duplex molecule. Nucleic acid hybridization techniques can be used to obtain an isolated nucleic acid within the scope of the disclosure. Briefly, any nucleic acid having some homology to the gene coding for a lipoate synthase, IscS, Nfs1, and/or IscU, can be used as a probe to identify a similar nucleic acid by hybridization under conditions of moderate to high stringency. Once identified, the nucleic acid then can be purified, sequenced, and analyzed to determine if it is the gene coding for a lipoate synthase, IscS, Nfs1 and/or IscU sequence having the desired activity (i.e. lipoate synthase activity or an ability to assemble or regenerate Fe—S clusters of lipoate synthase).

Hybridization can be done by Southern or Northern analysis to identify a DNA or RNA sequence, respectively, that hybridizes to a probe. The probe can be labeled, for example with a biotin, a fluorophore, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring Harbor Laboratory, Plainview, N.Y. Typically, a probe is at least about 20 nucleotides in length. For example, a probe including 20 contiguous nucleotides of a lipoate synthase gene sequence can be used to identify an identical or similar nucleic acid. In addition, probes longer or shorter than 20 nucleotides can be used.

The disclosure also provides isolated nucleic acid sequences that are at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and hybridize, under hybridization conditions, to the sense or antisense strand of a lipoate synthase, IscS, Nfs1, and/or IscU nucleic acid sequence. The hybridization conditions can be moderately or highly stringent hybridization conditions. Sequences that hybridize to one another, for example using the conditions disclosed herein, are referred to as homologs.

Moderately stringent hybridization conditions include hybridization at about 42° C. in a hybridization solution including 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), and washes performed at about 50° C. with a wash solution containing 2×SSC and 0.1% sodium dodecyl sulfate (SDS).

For high stringency, the same hybridization conditions can be used, but washes are performed at about 65° C. with a wash solution containing 0.2×SSC and 0.1% SDS.

Isolated: An “isolated” biological component (such as a nucleic acid or protein) has been substantially separated or purified away from other biological components in the cell of the organism in which the component naturally occurs, or away from other biological components in the supernatant in which the organism grows.

In one example, isolated refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

In one example, isolated includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

In a particular example, an isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a cDNA or genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid.

In another example, isolated ALA refers to ALA produced by a microorganism which has been separated away from the microorganism, such as extracted or removed from the intracellular portion of the microorganism, or which has been purified or concentrated from the supernatant into which the microorganism secreted the ALA.

Nucleic acid: Encompasses both RNA and DNA including, without limitation, mRNA, cDNA, genomic DNA, and synthetic (e.g., chemically synthesized) DNA and RNA. The nucleic acid can be double-stranded or single-stranded. Where single-stranded, the nucleic acid can be the sense strand or the antisense strand. In addition, nucleic acid can be circular or linear.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Polynucleotide: A linear nucleic acid sequence of any length. Therefore, a polynucleotide includes molecules which are at least about 15, 25, 50, 75, 100, 200 or 400 (oligonucleotides) and also nucleotides as long as a full-length cDNA.

Polypeptide: Any chain of amino acids, regardless of length or post-translational modification, that retains the function of the full-length polypeptide. For example, full-length lipoate synthase from E. coli is 281 amino acids in length and has an approximate molecular weight of 31.3 kDa. A fragment of this 281 amino acid sequence which retains lipoate synthase activity, is also a lipoate synthase polypeptide. In one example, polypeptides can include one or more amino acid derivatives, such as 4-hydroxyproline and 5-hydroxylysine.

Probes and primers: A “probe” includes a nucleic acid or polypeptide containing a detectable label or reporter molecule. Typical labels include radioactive isotopes, ligands, chemiluminescent agents, fluorophores, and enzymes. Methods for labeling and guidance in the choice of labels appropriate for various purposes are discussed in, for example, Sambrook et al. (ed.), Molecular Cloning: A Laboratory Manual 2nd ed., vol. 1-3, Cold Spring Harbor Laboratory Press, Cold Spring Harbor, N.Y., 1989, and Ausubel et al. (ed.) Current Protocols in Molecular Biology, Greene Publishing and Wiley-Interscience, New York (with periodic updates), 1987.

“Primers” are typically nucleic acid molecules having 10 or more nucleotides (e.g., nucleic acid molecules having between about 10 nucleotides and about 100 nucleotides). A primer can be annealed to a complementary target nucleic acid strand by nucleic acid hybridization to form a hybrid between the primer and the target nucleic acid strand, and then extended along the target nucleic acid strand by, for example, a DNA polymerase enzyme. Primer pairs can be used for amplification of a nucleic acid sequence, for example, by the polymerase chain reaction (PCR) or other nucleic-acid amplification methods known in the art.

Methods for preparing and using probes and primers are described, for example, in Sambrook et al. (ed.), Molecular Cloning: A Laboratory Manual, 2nd ed., vol. 1-3, Cold Spring Harbor Laboratory Press, Cold Spring Harbor, N.Y., 1989; Ausubel et al. (ed.), Current Protocols in Molecular Biology, Greene Publishing and Wiley-Interscience, New York (with periodic updates), 1987; and Innis et al., PCR Protocols: A Guide to Methods and Applications, Academic Press: San Diego, 1990. PCR primer pairs can be derived from a known sequence, for example, by using computer programs intended for that purpose such as Primer (Version 0.5, © 1991, Whitehead Institute for Biomedical Research, Cambridge, Mass.). One of skill in the art will appreciate that the specificity of a particular probe or primer increases with the length, but that a probe or primer can range in size from a full-length sequence to sequences as short as five consecutive nucleotides. Thus, for example, a primer of 20 consecutive nucleotides can anneal to a target with a higher specificity than a corresponding primer of only 15 nucleotides. Thus, to obtain greater specificity, probes and primers can be selected that include, for example, 10, 20, 25, 30, 35, 40, 50, 60, 70, 80, 90, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 2000, 2050, 2100, 2150, 2200, 2250, 2300, 2350, 2400, 2450, 2500, 2550, 2600, 2650, 2700, 2750, 2800, 2850, 2900, 3000, 3050, 3100, 3150, 3200, 3250, 3300, 3350, 3400, 3450, 3500, 3550, 3600, 3650, 3700, 3750, 3800, 3850, 3900, 4000, 4050, 4100, 4150, 4200, 4250, 4300, 4350, 4400, 4450, 4500, 4550, 4600, 4650, 4700, 4750, 4800, 4850, 4900, 5000, 5050, 5100, 5150, 5200, 5250, 5300, 5350, 5400, 5450, or more consecutive nucleotides.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Purified: The term “purified” does not require absolute purity; rather, it is intended as a relative term. Thus, for example, a purified protein, nucleic acid, or cell is one in which the subject protein, nucleic acid, or cell is at a higher concentration than the protein, nucleic acid, or cell in its natural environment, such as within a cell or within an organism. For example, a polypeptide preparation can be considered purified if the polypeptide content in the preparation represents at least 50%, 60%, 70%, 80%, 85%, 90%, 92%, 95%, 98%, or 99% of the total protein content of the preparation. In another example, purified refers to a purified or concentrated compound, such as ALA, in which the compound is at a higher purity or concentration than the compound is in the supernatant into which the compound is secreted by a cell. For example, a preparation can be considered purified if the compound in the supernatant has been concentrated by at least 2-fold, for example by at least 5-fold, 10-fold, 20-fold, 100-fold, or even 1000-fold.

Recombinant: A recombinant nucleic acid is one that has a sequence that is not naturally occurring and/or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. “Recombinant” is also used to describe nucleic acid molecules that have been artificially manipulated, but contain the same regulatory sequences and coding regions that are found in the organism from which the nucleic acid was isolated.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (e.g., human and mouse sequences), compared to species more distantly related (e.g., human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq-i c:\seq1.txt-j c:\seq2.txt-p blastn-o c:\output.txt-q-1-r 2.

To compare two amino acid sequences, the options of B12seq can be set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (i.e., 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75).

| | |
|---|---|
| | 1 20 |
| Target Sequence: | AGGTCGTGTACTGTCAGTCA |
| | \| \|\| \|\|\| \|\|\|\| \|\|\|\| \| |
| Identified Sequence: | ACGTGGTGAACTGCCAGTGA |

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, 80%, 85%, 90%, 95%, or 99% sequence identity.

When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 60%, 70%, 75%, 80%, 85%, 90%, 95%, 98%, 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions. Stringent conditions are sequence-dependent and are different under different environmental parameters. Nucleic acid molecules that hybridize under stringent conditions to a lipoate synthase, IscS, Nfs1, or IscU gene sequence typically hybridize to a probe based on either an entire lipoate synthase, IscS, Nfs1, or IscU gene or selected portions of the gene, respectively, under conditions described above.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, 70%, 80%, 90%, 95%, 98%, or 99% sequence identity determined by this method.

One of skill in the art will appreciate that these sequence identity ranges are provided for guidance only; it is possible that strongly significant homologs could be obtained that fall outside the ranges provided.

An alternative (and not necessarily cumulative) indication that two nucleic acid sequences are substantially identical is that the polypeptide which the first nucleic acid encodes is immunologically cross reactive with the polypeptide encoded by the second nucleic acid.

Subject: Living multicellular vertebrate organisms, a category which includes, both human and veterinary subjects for example, mammals, birds, rodents, and primates.

Supernatant: The culture medium in which a cell or microorganism is grown. The culture medium may include material from the cell or microorganism, such as products secreted by the cell or microorganism, for example ALA.

Therapeutically effective amount: An amount sufficient to achieve a desired biological effect. In one example, it is an amount effective to allow a desired effect in a subject being treated, or which is capable of relieving signs or symptoms caused by a pathological condition, or which is capable of preventing signs or symptoms caused by a pathological condition. Diseases include, but are not limited to, diabetes, glaucoma, liver disorders, cardiac disorders, viral infections such as HIV, poisoning, Lyme disease, neurological disorders such as Alzheimer's, and inflammation.

Transformed: A cell/microorganism into which a nucleic acid molecule has been introduced, for example by molecular biology techniques. As used herein, the term transformation encompasses all techniques by which a nucleic acid molecule might be introduced into such a cell or microorganism, including, but not limited to transfection with viral vectors, conjugation, transformation with plasmid vectors, and introduction of naked DNA by electroporation, lipofection, and particle gun acceleration.

Variants, fragments or fusion proteins: The production of proteins can be accomplished in a variety of ways. DNA sequences which encode for a protein, fusion protein, or a fragment or variant of a protein, can be engineered to allow the protein to be expressed in eukaryotic or prokaryotic cells, such as yeast, bacteria, fungi, insect, and/or plant cells. To obtain expression, a DNA sequence can be altered and operably linked to other regulatory sequences. The final product, which contains the regulatory sequences and the gene encoding the protein, is a vector. This vector can be introduced into the desired cells. Once inside the cell the vector allows the protein to be produced.

A fusion protein comprising a protein, such as a lipoate synthase, IscS, Nfs1, or IscU (or variant, homolog, polymorphism, mutant, or fragment thereof) linked to other amino acid sequences that do not inhibit the desired activity of lipoate synthase, IscS, Nfs1, or IscU, for example the ability of lipoate synthase to synthesize ALA from octanoic acid esters, and the ability of IscS, Nfs1, and IscU to assemble or regenerate Fe—S clusters of lipoate synthase. In one embodiment, the other amino acid sequences are no more than about 10, 20, 30, or 50 amino acids in length.

One of ordinary skill in the art will appreciate that a DNA sequence can be altered in numerous ways without affecting the biological activity of the encoded protein. For example, PCR can be used to produce variations in the DNA sequence which encodes a lipoate synthase, IscS, Nfs1, or IscU. Such variants can be variants optimized for codon preference in a host cell used to express the protein, or other sequence changes that facilitate expression.

Vector: A nucleic acid molecule as introduced into a cell or microorganism, thereby producing a transformed cell or microorganism. A vector may include nucleic acid sequences that permit it to replicate in the cell or microorganism, such as an origin of replication. A vector may also include one or more selectable marker genes and other genetic elements known in the art.

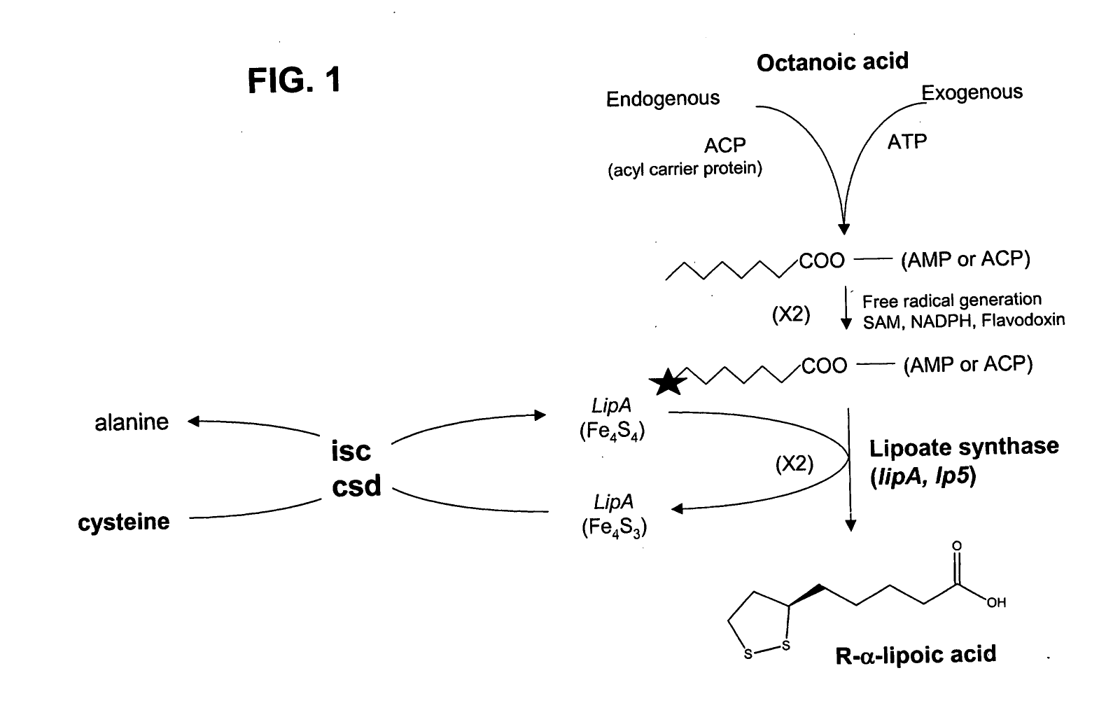

Herein disclosed are methods for producing ALA using engineered microorganisms, and particular, methods for producing non-racemic, enantiomerically pure R-ALA. ALA is synthesized from octanoic acid esters by lipoate synthase, an approximately 31-36 kDa enzyme that contains at least one iron-sulfur (Fe—S) cluster (FIG. 1). Fe—S clusters are involved in a number of cellular metabolic processes, and generally, are thought to serve as redox centers in electron transfer reactions, as catalytic centers in redox and nonredox reactions, and as sensors of iron, dioxygen, and superoxide in the regulation of metabolic pathways. As described herein, ALA can be produced in microorganisms at supraendogenous levels (i.e., >35 μg ALA per g dry cell weight in prokaryotes and >0.5 μg ALA per g dry cell weight intracellular or >8.7 μg ALA per g dry cell weight extracellular, in eukaryotes) by expressing a lipoate synthase polypeptide alone or in combination with one or more additional polypeptides that assemble or regenerate the Fe—S clusters of lipoate acid synthase (i.e., assemble or regenerate Fe₄S₄ clusters of lipoate synthase), such as IscS, IscU, Nfs1, and/or Csd, or homologs or variants thereof that retain the appropriate biological activity. ALA produced using the disclosed methods can be produced intracellularly in the microorganism, and in some examples, is secreted extracellularly.

### Polypeptides

The disclosure provides enzyme polypeptides, such as lipoate synthase, IscS, IscU, Csd, Nfs1 and variants (such as homologs), fragments, and fusions thereof that retain lipoate synthase activity, or the ability to assemble or regenerate Fe—S clusters of lipoate synthase, respectively. One skilled in the art will understand that variant sequences can be used, as long as the polypeptide retains the desired activity, such as lipoate synthase activity, or the ability to assemble or regenerate Fe—S clusters of lipoate synthase. For example, the disclosure provides polypeptides that contain at least 15 contiguous amino acids which are identical to an enzyme sequence, such as a lipoate synthase, an IscS, an Nfs1, and/or an IscU sequence which retain the appropriate biological activity. It will be appreciated that the disclosure also provides polypeptides that include an amino acid sequence that is greater than at least 15 amino acid residues (e.g., at least 16, 17, 18, 19, 20, 21, 22, 23, 24, 25, 26, 27, 28, 29, 30, 50, 75, 100, 150, 200, 250, 300 or more amino acid residues) in length and identical to any enzyme disclosed herein or otherwise publicly available.

In addition, the disclosure provides one or more enzymes, such as a lipoate synthase, an IscS, a Csd, an Nfs1, and an IscU, which include an arnino acid sequence having a variation of the enzyme amino acid sequence. Variant sequences can contain a single insertion, a single deletion, a single substitution, multiple insertions, multiple deletions, multiple substitutions, or any combination thereof (e.g., single deletion together with multiple insertions). Such polypeptides share at least 60, 65, 70, 75, 80, 85, 90, 95, 97, 98, or 99% sequence identity with an enzyme sequence, such as a lipoate synthase, an IscS, an IscI, and an IscU sequence, as long as the peptide encoded by the amino acid sequence retains the desired enzyme activity.

Polypeptides having a variant amino acid sequence can retain activity, such as lipoate synthase activity or the ability to assemble or regenerate Fe—S clusters of lipoate synthase. Such polypeptides can be produced by manipulating the nucleotide sequence encoding a polypeptide using standard procedures such as site-directed mutagenesis or PCR. One type of modification includes the substitution of one or more amino acid residues for amino acid residues having a similar biochemical property, that is, a conservative substitution. For example, lipoate synthase variants can be used to encode a polypeptide having lipoate synthase activity. For example, the following variations can be made to the lipoate synthase amino acid sequence shown in SEQ ID NO: 15: the Val at position 6 of can be substituted with an Ile or a Leu; the Thr at position 21 can be substituted with a Ser; and the Asn at position 43 can be substituted with a Gln or His. It will be appreciated that the sequence set forth in SEQ ID NO: 15 can contain any number of variations as well as any combination of types of variations, as long as the peptide retains lipoate synthase activity.

More substantial changes can be obtained by selecting substitutions that are less conservative, i.e., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed for polypeptides having biological activity by analyzing the ability of the polypeptide to catalyze the conversion of the same substrate as the related native polypeptide to the same product as the related native polypeptide. Accordingly, polypeptides having at least 1, 2, 5, 10, 20, 30, 40, 50 or more conservative substitutions are provided herein.

Polypeptides and nucleic acids encoding the polypeptide can be produced by standard DNA mutagenesis techniques, for example, M13 primer mutagenesis. Details of these techniques are provided in Sambrook et al. (ed.), Molecular Cloning: A Laboratory Manual 2nd ed., vol. 1-3, Cold Spring Harbor Laboratory Press, Cold Spring, Harbor, N.Y., 1989, Ch. 15. Nucleic acid molecules can contain changes of a coding region to fit the codon usage bias of the particular organism into which the molecule is to be introduced.

Alternatively, the coding region can be altered by taking advantage of the degeneracy of the genetic code to alter the coding sequence in such a way that, while the nucleic acid sequence is substantially altered, it nevertheless encodes a polypeptide having an amino acid sequence identical or substantially similar to the native amino acid sequence. For example, because of the degeneracy of the genetic code, alanine is encoded by the four nucleotide codon triplets: GCT, GCA, GCC, and GCG. Thus, the nucleic acid sequence of the open reading frame can be changed at an alanine position to any of these codons without affecting the amino acid sequence of the encoded polypeptide or the characteristics of the polypeptide. Based upon the degeneracy of the genetic code, nucleic acid variants can be derived from a nucleic acid sequence using a standard DNA mutagenesis techniques as described herein, or by synthesis of nucleic acid sequences. Thus, this disclosure also encompasses nucleic acid molecules that encode the same polypeptide but vary in nucleic acid sequence by virtue of the degeneracy of the genetic code.

### Lipoate Synthase Polypeptides

Suitable nucleic acids that encode lipoate synthase polypeptides have been cloned and sequenced from several organisms, including E. coli and S. cerevisiae. The amino acid sequences of the E. coli and S. cerevisiae lipoate synthase polypeptides are provided under GenBank Accession No. P25845 and GenBank Accession No. P32875, respectively, and the nucleic acid sequences are provided under GenBank Accession No. M82805 and GenBank Accession No. Z75104, respectively. Homologs of lipoate synthase can be identified as described below. Lipoate synthase polypeptides that are useful are those that retain the ability to synthesize ALA from octanoic acid esters, that is, they retain lipoate synthase activity.

### Fe—S Cluster Assembling or Regenerating Polypeptides

Suitable Fe—S cluster assembling or regenerating polypeptides include the nitrogen fixation polypeptides (Nif) or iron-sulfur cluster polypeptides (Isc) S (IscS) and U (IscU) of Azotobacter vinelandii, which are involved in the maintenance or generation of Fe—S clusters of nitrogenase and other polypeptides, and homologs of IscS and IscU polypeptides from other microorganisms. Fe—S cluster regenerating polypeptide activity is the ability to assemble or regenerate Fe—S clusters of an Fe—S cluster enzyme. For example, IscS and IscU activity is the ability of IscS and/or IscU to assemble or regenerate the Fe—S clusters of lipoate synthase.

Full-length A. vinelandii IscS and IscU polypeptides are 402 and 312 amino acids in length, respectively. IscS polypeptides are pyridoxal-phosphate dependent cysteine desulfurases that catalyze the reductive conversion of cysteine to alanine and sulfide. The sulfur is transiently bound to a specific cysteine side-chain of the enzyme and transferred into the Fe—S clusters by an unknown mechanism. IscU polypeptide is a homodimer that contains one 2Fe-2S cluster per subunit. IscU may deliver iron or may provide an intermediate site for Fe—S cluster assembly. The nucleic acids encoding IscS and IscU are found on an “nif” gene cluster in A. vinelandii. The nucleic acid and amino acid sequences of A. vinelandii IscS and IscU are provided under GenBank Accession No. M17349. The nucleic acid and amino acid sequence of S. cerevisiae Nfs1 are provided under GenBank Accession No. 10383748.

Homologs of the A. vinelandii IscS and IscU polypeptides have been identified in other species, including E. coli, B. subtilis, R. capsulatus, R. sphaeroides, Pseudomonas aeroginosa, Haemophilus influenzae, and in some examples, are found on an isc gene cluster. Non-limiting examples of IscS homologs include IscSI polypeptides (also may be called homocysteine desulfohydratases (EC 4.4.1.2)/selenocysteine lyase (4.4.1.16) or cysteine desulfurase), cysteine sulfinic desulfinase (Csd) polypeptides, and IscSII polypeptides (also may be called homocysteine desulfhydrase (EC 4.4.1.2)/selenocysteine lyase (4.4.1.16) or cysteine desulfurase). Gene clusters within E. coli, R. sphaeroides, R. capsulatus, vinelandii, P. aeruginosa, H. influenzae, and Klebsiella pneumoniae each encode a IscSI polypeptide, a IscU polypeptide, and a ferredoxin polypeptide. The gene clusters of E. coli, A. vinelandii, P. aeruginosa, H. influenzae, and K. pneumoniae also contain a polypeptide that shows sequence similarity to previously identified transcriptional regulators. This polypeptide has been designated ORF 2 or iscR in the E. coli gene cluster and may negatively regulate the expression of the gene cluster. Csd polypeptides are pyridoxal-phosphate dependent enzymes that catalyze the desulfuration of L-cysteine, L-selenocysteine, L-selenocystine, L-cystine, and cysteine sulfinic acid. IscSI, IscSII, and Csd polypeptides also are classified as class-V-pyridoxal phosphate dependent aminotransferases.

The nucleic acid sequences encoding IscSI, Csd, and IscSII polypeptides, as well as the amino acid sequences of such polypeptides are available in GenBank, at the Department of Energy Joint Genome Institute (DOE/JGI) web site or at the ERGO website. For example, see GenBank Accession No. Q07177 for the amino acid sequence of a IscSI polypeptide from R. capsulatus; GenBank Accession No. X68444 for the nucleic acid sequence encoding IscU and IscS from R. capsulatus; GenBank Accession No. Q01179 for the amino acid sequence of a IscSI polypeptide from R. sphaeroides; GenBank Accession No. M86823 for the nucleic acid sequence encoding IscS and IscU from R. sphaeroides; GenBank Accession No. P39171 for the amino acid sequence of a IscSI polypeptide from E. coli; and GenBank Accession No. P38033 for the sequence of a IscSI from B. subtilis. See, GenBank Accession No. CAB77085 for a Csd from E. coli; and contig 735 23,481 to 22,228 of ERGO web site for the sequence of a Csd from Yersinia pestis. See, GenBank Accession No. CAC07717 for the sequence of a IscS-II from E. coli; GenBank Accession No. 032164 for a IscSII sequence from B. subtilis; contig 2A12-2D05, 73,689 to 71,914 of the ERGO web site for IscSII sequences from R. capsulatus; and contig 0063, 58,864 to 57,647 of the DOE/JGI web site for IscSII sequences from R. sphaeroides. The nucleic acid and amino acid sequence of S. cerevisiae Nfs1 are provided under GenBank Accession No. 10383748.

Other homologs of IscS or IscU can be identified by sequence comparisons. For example, the nucleic acid or amino acid sequences of the putative homologs and the nucleic acid or amino acid sequences of IscS or IscU can be compared by aligning the sequences and calculating percent identity. Generally, percent sequence identity is calculated by determining the number of matched positions in aligned amino acid sequences, dividing the number of matched positions by the total number of aligned amino acid residues, and multiplying by 100. A matched position refers to a position in which identical amino acids occur at the same position in aligned amino acid sequences.

### Nucleic Acid Molecules

The nucleic acid sequences encoding the polypeptides disclosed herein, such as lipoate synthase, IscS, Csd, Nfs1 and IscU (as well as any other sequence disclosed herein), can contain an entire nucleic acid sequence encoding the protein, as well as a portions thereof that retain the desired activity. For example, a nucleic acid can include at least 15 contiguous nucleotides of a full-length cDNA sequence. The disclosure also provides isolated nucleic acids which include a nucleotide sequence that is greater than 12 nucleotides (e.g., at least 15, 16, 17, 18, 19, 20, 21, 22, 23, 24, 25, 26, 27, 28, 29, 30, 40, 50, 75, 100 or more nucleotides) in length and identical to any portion of an enzyme sequence, such as a lipoate synthase, IscS, Csd, Nfs1, and/or IscU sequence. In some examples, the isolated nucleic acid molecules encode a full-length polypeptide, i.e., a full-length lipoate synthase or polypeptide that assembles or regenerates Fe—S clusters of lipoate synthase. Nucleic acid molecules can be DNA or RNA, linear or circular, and in sense or antisense orientation.

In addition, the disclosure provides isolated nucleic acid sequences which contain a variation of a nucleic acid sequence, such as a variant lipoate synthase, IscS, Csd, Nfs1, and/or IscU nucleic acid sequence. Variants can contain a single insertion, a single deletion, a single substitution, multiple insertions, multiple deletions, multiple substitutions, or any combination thereof (e.g., single deletion together with multiple insertions). Such isolated nucleic acid molecules can share at least 60, 65, 70, 75, 80, 85, 90, 92, 95, 97, 98, or 99% sequence identity with an enzyme sequence, such as a lipoate synthase, IscS, Csd, Nfs1, and/or IscU sequence, as long as the peptide encoded by the nucleic acid retains the desired enzyme activity, such as lipoate synthase activity or the ability to assemble or regenerate Fe—S clusters of lipoate synthase. For example, lipoate synthase variants can be used to encode a polypeptide having lipoate synthase activity. For example, the following variations can be made to the lipoate synthase nucleic acid sequence shown in SEQ ID NO: 14: the “t” at position 15 can be substituted with a “g” or “c”; the “t” at position 63 can be substituted with an “a” or “c”; the “c” at position 129 can be substituted with a “t”; and the “a” at position 489 can be substituted with a “t”, “g”, or “c”. It will be appreciated that the sequence set forth in SEQ ID NO: 14 can contain any number of variations as well as any combination of types of variations, as long as the peptide retains lipoate synthase activity.

Codon preferences and codon usage tables for a particular microorganism can be used to engineer isolated nucleic acid molecules that take advantage of the codon usage preferences of that particular species of microorganism. For example, the sequences disclosed herein can be designed to have codons that are preferentially used by a particular organism of interest.

The disclosure also provides isolated nucleic acid sequences that encode for an protein, such as lipoate synthase, IscS, Csd, Nfs1, and/or IscU wherein the sequence is at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and hybridizes, under hybridization conditions, to the sense or antisense strand of a nucleic acid encoding the enzyme. The hybridization conditions can be moderately or highly stringent hybridization conditions.

Nucleic acids encoding lipoate synthase polypeptides or polypeptides that assemble or regenerate Fe—S clusters of lipoate synthase can be obtained or produced using any method including, without limitation, common molecular cloning and chemical nucleic acid synthesis techniques. For example, polymerase chain reaction (PCR) techniques can be used. PCR refers to a procedure or technique in which target nucleic acids are amplified. Sequence information from the ends of the region of interest or beyond typically is employed to design oligonucleotide primers that are identical to in sequence, or have a high degree of identity, to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are known (PCR Primer: A Laboratory Manual, Ed. by Dieffenbach, C. and Dveksler, G., Cold Spring Harbor Laboratory Press, 1995). When using RNA as a source of template, reverse transcriptase can be used to synthesize complimentary DNA (cDNA) strands.

Nucleic acids encoding lipoate synthase polypeptides or polypeptides that assemble or regenerate Fe—S clusters also can be chemically synthesized, either as a single nucleic acid molecule or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a double-stranded nucleic acid molecule per oligonucleotide pair, which then can be ligated into a vector.

Mutagenesis also can be used to obtain nucleic acids encoding particular lipoate synthase polypeptides or polypeptides that assemble or regenerate Fe—S clusters of lipoate synthase. For example, a nucleic acid encoding lipoate synthase, IscS, Csd, Nfs1, or IscU can be mutated using common molecular cloning techniques (e.g., site-directed mutagenesis). Possible mutations include, without limitation, deletions, insertions, and substitutions, as well as combinations of deletions, insertions, and substitutions. Alignment of lipoate synthase, IscS, Csd, Nfs1, or IscU sequences described above can be used to identify positions to modify, e.g., to identify which nucleotides can be substituted, which nucleotides can be deleted, and at which positions nucleotides can be inserted.

In addition, nucleic acid and amino acid databases (e.g., GenBank) can be used to obtain nucleic acids encoding lipoate synthase or Fe—S cluster assembling or regenerating polypeptides. For example, any nucleic acid sequence or amino acid sequence having homology to a lipoate synthase, IscS, Csd, Nfs1 or IscU sequence described above can be used as a query to search GenBank.

Furthermore, nucleic acid hybridization techniques can be used to obtain nucleic acid homologs which encode lipoate synthase and polypeptides that assemble or regenerate Fe—S clusters. Briefly, any nucleic acid having homology to a sequence described above can be used as a probe to identify a similar nucleic acid by hybridization under conditions of moderate to high stringency.

Once a nucleic acid is identified, the nucleic acid then can be purified, sequenced, and analyzed to determine whether it is within the scope of the disclosure as described herein. Hybridization can be done by Southern or Northern analysis to identify a DNA or RNA sequence, respectively, that hybridizes to a probe. The probe can be labeled with biotin, digoxygenin, an enzyme, or a radioisotope such as ³²P or ³⁵S. The DNA or RNA to be analyzed can be electrophoretically separated on an agarose or polyacrylamide gel, transferred to nitrocellulose, nylon, or other suitable membrane, and hybridized with the probe using standard techniques well known in the art (Sambrook et al., 1989, Molecular Cloning, second edition, Cold Spring harbor Laboratory, Plainview, N.Y., Sections 7.39-7.52).

### Nucleic Acid Constructs

Nucleic acid constructs include an expression control element such as a promoter operably linked to a nucleic acid having a sequence encoding a lipoate synthase polypeptide and/or a nucleic acid including a sequence encoding a polypeptide that assembles or regenerates the Fe—S clusters of a lipoate synthase. For example, a nucleic acid construct may contain sequences that encode one or more of the following polypeptides: lipoate synthase, IscSI, IscSII, IscU, Csd, Nfs1, and homologs. Expression control elements do not typically encode a gene product, but instead affect the expression of the nucleic acid sequence. Expression control elements can include, for example, promoter sequences, enhancer sequences, response elements, polyadenylation sites, or inducible elements. Non-limiting examples of promoters for bacterial expression include the lac promoter, trp promoter, combination of lac and trp (referred to as trc and tac, Amersham Pharmacia, Piscataway, N.J.), ara promoter (Clontech, Palo Alto, Calif.), phage lambda promoters, phage T7 promoter, and the phage T5 promoter (Qiagen, Valencia, Calif.). Non-limiting examples of promoters for yeast expression include AOX1, GAP, AUG1, GAL1, TEF1, nmtl, nmt41, and nmt81 (Invitrogen, Carlsbad, Calif.). Promoters for expression in higher eukaryotic cells include the ecdysone-inducible system, T-rex system, and Geneswitch system (Invitrogen, Carlsbad, Calif.).

Inducible promoters can be used to allow the production of ALA to be regulated. Inducible refers to both up-regulation and down regulation. An inducible promoter is a promoter capable of directly or indirectly activating transcription of one or more nucleic acid sequences or genes in response to an inducer. In the absence of an inducer, the nucleic acid sequences or genes are transcribed at a low level or are not transcribed. Examples of inducers include, but are not limited to: a chemical agent such as a protein, metabolite, growth regulator, chemical compound, such as a steroid; a physiological stress imposed directly by heat, cold, salt, or toxic elements, or indirectly through the action of a pathogen or disease agent such as a virus; an illumination agent such as light, darkness and lights various aspects, which include wavelength, intensity, fluorescence, direction, and duration.

Examples of inducible promoters include, but are not limited to: the lac system and the tetracycline resistance system from E. coli. In one version of the lac system, expression of lac operator-linked sequences is constitutively activated by a fusion protein and is activated in the presence of IPTG. In another version of the Lac system, expression of lac-promoter/operator linked sequences leads to the expression of T7 RNA polymerase, an enzyme that transcribes DNA starting at a specific T7 promoter. Thus, sequences operably linked to the T7 promoter are transcribed when expression of the lac-controlled T7 RNA polymerase is induced by IPTG Components of the tetracycline (Tc) resistance system also can be used to regulate gene expression. For example, the Tet repressor (TetR), which binds to tet operator sequences in the absence of tetracycline and represses gene transcription, can be used to repress transcription from a promoter containing tet operator sequences.

Disclosed nucleic acid constructs can include a nucleic acid encoding a selectable marker. For example, selectable markers that provide antibiotic resistance (e.g., to ampicillin, kanamycin, tetracycline, streptomycin, or hygromycin) can be used. Such markers are useful for selecting transformants that contain a nucleic acid construct of the disclosure.

### Engineered Microorganisms

Microorganisms that are suitable for producing ALA include prokaryotic and eukaryotic microorganisms, such as bacteria, yeast, plants, algae, and fungi. For example, yeast such as Phaffia rhodozyma (Xanthophyllomyces dendrorhous), Candida utilis, C. lipolytica, and S. cerevisiae; fungi such as Neurospora crassa, Phycomyces blakesleeanus, Blakeslea trispora, and Aspergillus sp; Archaea such as Halobacterium salinarium, and Eubacteria including Pantoea species (formerly called Erwinia) such as Pantoea stewartii (e.g., ATCC Accession #8200), flavobacteria species such as Xanthobacter autotrophicus and Flavobacterium multivorum, Zymonomonas mobilis, Rhodobacter species such as R. sphaeroides and R. capsulatus, as well as Escherichia, for example E. coli and E. vulneris, can be used. Other examples of bacteria that can be used, include, but are not limited to bacteria in the genus Sphingomonas, Acetobacter, Clostridium, Bacillus, and Gram negative bacteria in the α-subdivision, such as Paracoccus, Azotobacter, Agrobacterium, and Erythrobacter. Acid tolerant bacteria in the genus, for example, Lactobacillus and Gluconobacter also can be used. R. sphaeroides and R. capsulatus grow on defined media and are non-pyrogenic, minimizing health concerns about use in nutritional supplements. In some examples, ALA is produced in plants or algae such as Haematococcus pluvialis, Dunaliella salina, Chlorella protothecoides, Zea mays, Arabidopsis thaliana, Glycine max, and Neospongiococcum excentricum.

In general, the engineered microorganisms disclosed herein are produced by introducing one or more exogenous nucleic acids (e.g., a nucleic acid construct described above) into the microorganisms by standard methodologies, including, but not limited to, calcium phosphate precipitation, conjugation, electroporation, heat shock, lipofection, microinjection, and viral-mediated nucleic acid transfer. In addition, naked DNA can be delivered directly to microorganisms in vivo as described elsewhere (e.g. U.S. Pat. Nos. 5,580,859 and 5,589,466). Engineered microorganisms can be stably or transiently transfected, i.e., the exogenous nucleic acid can be integrated into the genome of the microorganism or maintained in an episomal state.

Disclosed microorganisms can contain a single copy or multiple copies (e.g., about 5, 10, 20, 35, 50, 75, 100 or 150 copies), of a particular exogenous nucleic acid. In addition, microorganisms can contain more than one particular exogenous nucleic acid. For example, a microorganism can contain about 15 copies of exogenous nucleic acid X as well as about 75 copies of exogenous nucleic acid Y. In these cases, each different nucleic acid can encode a different polypeptide having its own unique enzymatic activity (e.g., ability of lipoate synthase to synthesize ALA from octanoic acid esters or of polypeptides to assemble or regenerate Fe—S clusters of lipoate synthase). For example, a microorganism can contain two different exogenous nucleic acids such that a high level of ALA is produced. Alternatively, a single exogenous nucleic acid can encode one or more polypeptides. For example, a single nucleic acid can contain sequences that encode two or more different polypeptides. In either case, microorganisms can include one or more exogenous nucleic acids such that lipoate synthase is expressed alone or in combination with one or more polypeptides that assemble or regenerate the Fe—S clusters of lipoate synthase. One or more of the following polypeptides may be expressed in microorganisms disclosed herein: lipoate synthase, IscSI, IscSII, IscU, and Csd, or Nfs1. Suitable nucleic acids encoding these polypeptides are described above. As described herein, expressing lipoate synthase, IscS, and IscU or lipoate synthase and Csd produces greater than 200 μg ALA per g dry cell weight in microorganisms, such as-prokaryotes. Expressing lipoate synthase and/or Nfs1 produces greater than 1 μg ALA per g dry cell weight of intracellular ALA and greater than 7 μg ALA per g dry cell weight extracellular ALA in microorganisms, such as yeast.

In some examples, one or more exogenous nucleic acids (e.g., lipoate synthase) are introduced into a microorganism that is lacking the transcriptional regulator ORF such as the iscR gene in E. coli. The transcription regulator ORF down-regulates the Fe—S gene cluster assembly. Thus, expressing lipoate synthase in a microorganism lacking the transcriptional regulator ORF can lead to production of large quantities of ALA. Microorganisms lacking a transcriptional regulator ORF can be produced using common mutagenesis, knock-out, or antisense techniques. For example, a gene replacement strategy can be used (e.g. Datsenko and Wanner, Proc. Natl. Acad. Sci. USA 97: 6640-5, 2000). The iscR region can be deleted and an antibiotic resistance cartridge inserted into its place in the chromosome by homologous recombination. The antibiotic resistance cartridge can, in turn, be excised by a subsequent recombination event using the FLP function encoded by plasmid pCP20, as described by Datsenko and Wanner (Proc. Natl. Acad. Sci. USA 97: 6640-5, 2000).

Microorganisms containing exogenous nucleic acids can be identified by any method. For example, PCR and nucleic acid hybridization techniques such as Northern and Southern analysis can be used. In some cases, immunohistochemistry and biochemical techniques can be used to determine if a microorganism contains a particular nucleic acid by detecting expression of a polypeptide (e.g., lipoate synthase or a polypeptide that assembles or regenerates the Fe—S clusters of lipoate synthase). For example, the polypeptide of interest can be detected with an antibody having specific binding affinity for that polypeptide, which indicates that that cell not only contains the introduced nucleic acid but also expresses the encoded polypeptide. Enzymatic activities of the polypeptide of interest also can be detected (e.g., assembly or regeneration of Fe—S clusters or production of ALA) as an indication that the microorganism contains the introduced nucleic acid and expresses the encoded polypeptide from that introduced nucleic acid.

In one example, microorganisms of the disclosure produce at least 200 μg ALA per g dry cell weight, such as at least 500, 1000, 2000, 2500, 2600, 3000, 3500, 3900, 4000, 4500, 5000, 10,000, 15,000, 20,000, 25,000, 30,000, 40,000, 50,000 or even 100,000 μg ALA per g dry cell weight, at least including free and bound forms of ALA as well as oxidized and reduced forms of ALA. For example, the disclosed microorganisms can produce 40 to 100,000 μg ALA per g dry cell weight, 200 to 4000, 200 to 100,000 μg ALA per g dry cell weight, 500 to 75,000 μg ALA per g dry cell weight, 1,000 to 5,000 μg ALA per g dry cell weight, 1,000 to 10,000 μg ALA per g dry cell weight, 1,000 to 20,000 μg ALA per g dry cell weight, 2,500 to 4,000 μg ALA per g dry cell weight, 5000 to 50,000 μg ALA per g dry cell weight, or 10,000 to 30,000 μg ALA per g dry cell weight. Examples of such microorganisms include prokaryotes, such as E. coli. In a particular example, such ALA is produced intracellularly.

In another example, microorganisms of the disclosure produce at least 0.5 μg ALA per g dry cell weight intracellularly, such as at least 0.7, 1, 1.1, 1.5, 2, 5, 10, 100, 1000, 2000, 5000, 10,000, 15,000, 20,000, 25,000, 30,000, 40,000, 50,000 or even 100,000 μg ALA per g dry cell weight, at least including free and bound forms of ALA as well as oxidized and reduced forms of ALA. In another example, microorganisms of the disclosure secrete at least 7 μg ALA per g dry cell weight, such as at least 7.5, 8, 9, 10, 20, 25, 50, 75, 100, 200, 500, or even 1000 μg ALA per g dry cell weight, at least including free and bound forms of ALA as well as oxidized and reduced forms of ALA. Examples of such microorganisms include eukaryotes, such as yeast, for example S. cerevisiae. In a particular example, such ALA is produced intracellularly and secreted extracellularly. In another example, the amount of ALA secreted into the supernatant is at least 2-fold greater than the amount of ALA found intracellularly, such as at least 5-fold, 7-fold, 10-fold, 20-fold, or even 100-fold greater.

### Production of ALA in Microorganisms

Typically, ALA is produced by providing an engineered microorganism described above and culturing the microorganism under appropriate conditions. In general, the culture media and/or culture conditions can be such that the microorganisms grow to an adequate density and produce ALA efficiently.

For large-scale production processes, any method can be used such as those described elsewhere (Manual of Industrial Microbiology and Biotechnology, 2^{nd} Edition, Editors: Demain and Davies, ASM Press; and Principles of Fermentation Technology, Stanbury and Whitaker, Pergamon). Briefly, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated either in the presence or absence of oxygen to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank. For example, the first tank can contain medium with xylose, while the second tank contains medium with glucose.

Once transferred, the microorganisms can be incubated in the presence or absence of oxygen to allow for the production of ALA. Once produced, any method can be used to isolate the formed product, from the microorganism if the ALA is produced intracellularly, or from the broth if the ALA is secreted from the microorganism. For example, common separation techniques can be used to remove the biomass from the broth, and common isolation procedures (e.g., extraction, distillation, and ion-exchange procedures) can be used to obtain the ALA from the microorganism-free broth. Alternatively, the product can be isolated while it is being produced, or it can be isolated from the broth after the product production phase has been terminated. Additional detection methods are disclosed below.

### ALA Production

ALA can be produced by continuing growth of the microorganism in culture medium, or can be produced by adding exogenous octanoic (caprylic) acid to the culture. Octanoic acid can be added as a mixture in a co-solvent or in a solvent that forms the non-aqueous phase of a two-phase fermentation or bioconversion system. In one example, the concentration of octanoic acid does not exceed the concentration of octanoic acid at which metabolic activity of the microorganism is substantially inhibited or prevented. Once produced, any method can be used to isolate ALA. For example, the biomass can be collected and ALA can be released by treating the biomass or ALA can be extracted directly from the biomass.

Once isolated, ALA can be used directly, or can be formulated for various uses, including as a food additive, feed additive, pet food additive, nutritional supplement, nutraceutical, or a pharmaceutical. Thus, one or more additional components can be added to the isolated ALA, depending on the desired use, including, but not limited to vitamins, carotenoids, antioxidants such as ethoxyquin, vitamin E, butylated hydroxyanisole (BHA), butylated hydroxytoluene (BHT), or ascorbyl palmitate, vegetable oils such as corn oil, safflower oil, sunflower oil, or soybean oil, or an edible emulsifier, such as soy lecithin or sorbitan esters, or other pharmaceutical carrier. Addition of antioxidants and vegetable oils can help prevent degradation of the ALA during processing, shipment, and storage.

Alternatively, the biomass can be collected and dried, without extracting the ALA. The biomass then can be formulated for various uses as described below.

The amount of ALA produced by the disclosed engineered microorganisms, such as the amount of ALA per g dry cell weight or the amount of ALA produced intracellularly versus secreted, can be determined at a time following expression of one or more exogenous nucleic acid sequences, such as a lipoic acid synthase sequence and/or a sequence which encodes a polypeptide that assembles or regenerates an Fe—S cluster of the lipoic acid synthase polypeptide. The amount of time following expression can depend on, for example, the microorganism or cell used, the promoters used, growth temperature, and/or medium composition. For example, the amount of ALA per g dry cell weight produced by a disclosed microorganism can be an amount produced after at least: 10 minutes, 30 minutes, 45 minutes, 1 hour, 1.5 hours, 2 hours, 3, hours, 4 hours, 8 hours, 10 hours, 15 hours, 17 hours, 20 hours, 24 hours, 30 hours, 36 hours, 44 hours, 48 hours, 60 hours, 72 hours, 5 days, 1 week, 2 weeks, or 1 month of expression of one or more exogenous nucleic acid sequences. Such time-frames are not exclusive, and one skilled in the art will understand that other periods of time can be used.

### Methods of Detecting ALA Produced by a Microorganism

Standard analytical methodology can be used to quantitate the amount of ALA produced by a microorganism. For example, protein bound ALA can be quantified by gas chromatography with flame ionization detection and flame photometric detection after acid or base hydrolysis of the microorganism. Free ALA can be detected by high-performance liquid chromatography (Katoaoka, J. Chromatogr. B. Biomed. Sci. Appl. 717:247-62, 1998). In addition, as described herein, a bioassay can be used to estimate the amount of ALA produced by the microorganism. In general, growth of a strain of E. coli that requires lipoic acid for growth (such as the K12 strain, American Type Culture Collection Accession No. 25645, or the E. coli lipA::CAT strain described in EXAMPLE 5) can be assessed in the presence of varying amounts of a reconstituted extract of the engineered microorganism and compared to growth in the presence of known amounts of ALA.

The disclosure is further described in the following examples, which do not limit the scope of the disclosure.

### EXAMPLE 1

### Lipoic Acid Synthesis in E. coli

### Construction of Plasmids

The lipA, iscS-I, iscS-II, and csd genes and the iscS-IliscU gene cluster were obtained by PCR amplification of genomic DNA (isolated from E. coli strain M61655). Primers used for amplifying lipA were 5′-GGAACACGCACGCCATGGGTAAACCCATTGTG-3′(SEQ ID NO: 1, forward primer, a NcoI restriction site underlined) and 5′-GATGTAAGTAATTACTGCAGGATTACTTAA-3′(SEQ ID NO: 2, reverse primer, a PstI site underlined). The primers used for iscS-I amplification were 5′-GATTCCTTGCATCATATGATGTACGGAGTTT-3′(SEQ ID NO: 3, forward primer, NdeI site underlined) and 5′-TCCGATTCCGATCTAGATTAATGATGAGCCCATTC-3′(SEQ ID NO: 4, reverse primer, XbaI site underlined). The primers used for iscS-II amplification were 5′-CAGGAGGTGCCATATGATTTTTTCCGTCGACAA-3′(SEQ ID NO: 5, forward primer, NdeI site underlined) and 5′-CCATAGTGCCTCTAGATTATCCCAGCAAACGTGA-3′(SEQ ID NO: 6, reverse primer, XbaI site underlined). The primers used for csd amplification were 5′-GCCGAGGAGTCATATGAACGTTTTTAATC-3′(SEQ ID NO: 7, forward primer, NdeI site underlined) and 5′-GCGGGTTCTAGATTAATCCACCAATAATTC-3′(SEQ ID NO:8, reverse primer, XbaI site underlined). The primers used for the iscS-I/iscU gene cluster amplification were 5′-GATTCCTTGCATCATATGATGTACGGAGTT-3′ (SEQ ID NO: 3, forward primer) and 5′-ACCAAATCTAGAACTCTTATTTTGCTTCACGTTTG-3′ (SEQ ID NO: 9, reverse primer, XbaI site underlined). All primers were purchased from Integrated DNA Technologies, Inc. (Coralville, Iowa). PCR was carried out in an Eppendorf Mastercycler Gradient thermalcycler, using the Expand High Fidelity PCR system (Roche) under conditions recommended by the manufacturer. Optimal conditions were found to be 45° C. with 2 mM MgCl₂. PCR conditions were as follows: 96° C. for 5 minutes; followed by 10 cycles of 96° C. for 30 seconds, 45° C. for 60 seconds, and 72° C. for 105 seconds; then 15 cycles of 96° C. for 30 seconds, 45° C. for 60 seconds, and 72° C. for 105 seconds increasing 5 seconds per cycle; then 10 cycles of 96° C. for 30 seconds, 45° C. for 60 seconds, and 72° C. for 165 seconds; then 72° C. for 7 minutes.

The lipA PCR product was cloned into the NcoI/PstI sites of the pTRC99A vector (Amersham Pharmacia Biotech), while the iscS-I, iscS-II, csd, and iscS-I/iscU PCR products were cloned into the NdeI/XbaI sites of the pPRONde vector. The pPRONde vector is a derivative of the pPROLAR.A122 vector (Clontech Laboratories, Inc), in which an NdeI site has been introduced at bp 132 by site-directed mutagenesis. Ligations were carried out using the Rapid DNA ligation kit from Roche. Sequences of the inserts were verified by dideoxynucleotide chain-termination sequencing.

### Transformation and Expression of Proteins

Transformations were performed by electroporating 25 μL of E. coli DH10B ElectroMAX cells (Life Technologies, Inc., Gaithersburg, Md.) using the BioRad Gene Pulser II system according to manufacturers s recommendations (0.2 cm micro-electroporation cuvettes, 2.5 kV, 25 μFarads, and 200 ohms). Transformed cells were cultivated in 50 mL LB medium [tryptone (10 g/L), yeast extract (5 g/L), sodium chloride (10 g/L), pH 7.0, containing 100 μg/mL ampicillin (pTRC99a constructs) and/or 50 μg/mL kanamycin (pPRONde constructs)). The cells were grown at 37° C. with shaking (200 rpm) to an absorbance of between 0.5 and 0.8 at 650 nm, at which time protein expression was induced with 1 mM isopropyl-1-thio-galactopyranoside (IPTG). Cells were harvested (3,000×g for 20 min, 4° C.) after a further 4 hours of growth at 30° C. Cell pellets were re-suspended in 0.5 mL of 50 mM Tris-HCl, pH 7.0, and pelleted again by centrifugation. The washed pellets were resuspended in 50 μL lysis buffer (120 mM Tris-HCl, pH 6.8, 10% SDS, 10% 2-mercaptoethanol, 20% glycerol) and then incubated at 95° C. for 10 minutes. Any residual solid material was removed by centrifugation at 10,000×g for 5 minutes and 50 μL of H₂O were added to each of the cleared supernatants. Aliquots (5 to 10 μL) were analyzed for the level of protein expression by electrophoresis through pre-cast SDS-PAGE gels (4-15% and 12%) (BioRad). Expressed proteins represented approximately 2 to 20% of total protein.

### EXAMPLE 2

### Assay For ALA Production

A biological assay using a strain of E. coli that requires lipoic acid for growth was used to assess ALA production. One of two strains was used: the K12 strain, American Type Culture Collection Accession No. 25645, or the lipA::CAT strain described in EXAMPLE 5. A modification of the biological assay developed by Herbert and Guest, (Methods Enzymol. 18A:209-72, 1970) and Jackman et al. (Eur. J. Biochem. 193:91-5, 1990) was employed. In general, an inoculum of the lipoic acid requiring E. coli cells was obtained by growing the cells in culture medium containing ALA, then harvesting and washing the cells to remove ALA from the culture medium. Growth of the inoculum of the lipoic acid requiring E. coli cells was assessed in the presence of varying amounts of a reconstituted extract of the engineered DH10B cells containing one or more constructs described in EXAMPLE 1.

Reconstituted extracts of the engineered E. coli DH10B cells were prepared as follows. Cells were grown in 50 mL LB medium and induced as described in EXAMPLE 1. After 4 additional hours of growth, each cell culture was divided into two aliquots of 10 mL and 40 mL. The 40 mL aliquots were harvested by centrifugation and washed twice with 5 mL of 25 mM potassium phosphate buffer (pH 7.0). The washed pellets were re-suspended in 1 mL of 1 M HCl and incubated at 45° C. for 1 to 2 hours. After cooling, the suspensions were extracted two times with 0.5 mL methylene chloride, centrifuged, and the combined methylene chloride layers were dried under vacuum. The residues were dissolved in 25 mM potassium phosphate, pH 7.0. The smaller aliquot was used for determination of dry cell weight (DCW). After centrifugation at 3,000×g for 20 minutes (4° C.), the pellets were re-suspended in 1 mL H₂O, transferred to dried and pre-weighed tubes, and pelleted again by centrifugation at 10,000×g for 3 minutes. The H₂O wash was repeated once and the twice-washed pellets were dried to constant weight.

An inoculum was prepared by transferring a single colony of the the lipoic acid requiring E. coli to 5 mL of succinate-based, minimal salts medium supplemented with lipoic acid (1 ng/mL). Succinate-based, minimal salts medium contains 35 mM K₂HPO₄, 16 mM NaH₂PO₄, 37 mM NH₄Cl, 1 mM Na₂SO₄, 5 mg/mL thiamine, 0.5 mM MgCl₂, and 50 mM sodium succinate. Tubes were incubated at 37° C. with shaking for 24 hours. The cells were harvested by centrifugation for 10-20 minutes at 3,000×g, washed 2-3 times in sterile 0.9% (m/v) saline, and resuspended in saline to an absorbance at 650 nm of about 0.2.

The biological assay was performed by adding basal growth medium containing 50 mM succinate (1.0 mL), aliquots of the reconstituted extracts (sterile filtered with 0.2 μm filters ), and sufficient sterile water to each of a series of capped tubes (10×750 cm) for a total of 1.8 ml per tube. Basal growth medium (pH 6.8) contains per liter: 4 g acid-hydrolyzed casein (vitamin free), 14 g K₂HPO₄, 6 g KH₂PO₄, 1 g sodium citrate trihydrate, 0.2 g MgSO₄-7H₂O, 2 g (NH₄)₂SO₄, 2 g L-asparagine, 2 g L-arginine, 2 g L-glutamate, 2 g L-glycine, 2 g L-histidine, 2 g L-proline, 2 g L-tryptophan, 2 g L-cysteine, and 2 g sodium thioglycolate. The inoculum (0.02 mL) was added to each tube aseptically, and the tubes were incubated for approximately 44 hours at 37° C. with shaking. Bacterial growth was assessed by measuring the absorbance at 650 nm of the medium in each tube. A standard curve was generated in the same manner, substituting known quantities of ALA (0.2 to 2.0 ng) for the reconstituted sample extracts. The ALA content was calculated by comparison with the standard curve. All samples, including the standards, were set up in duplicate for each assay.

Table 1 provides the specific activity of each of the extracts, reported as μg ALA per g dry cell weight. As indicated in Table 1, LipA in combination with Csd, or in combination with IscSI and IscU, resulted in the production of approximately 2600 μg ALA per g dry cell weight and approximately 3900 μg ALA per g dry cell weight, respectively. These results demonstrate that engineered microorganisms can be used to produce levels of ALA which exceed endogenous levels (<35 μg ALA per g dry cell weight in prokaryotes), for example at least 200 μg ALA per g dry cell weight in prokaryotes. Furthermore, the disclosed methods allow the production of at least 200 μg ALA per g dry cell weight in prokaryotes of the R form of ALA, which is the active form found in enzymes, without the need for chemical synthesis.

TABLE 1
Production of ALA in E. coli

| Cloned gene/vector in E. coli DH10B cells | ALA content* |
|---|---|
| pPRONde (vector alone) | 0.5 |
| lipa/pTRC99A | 4 |
| lipA/pTRC99A + iscSI/pPRONde | 19 |
| lipA/pTRC99A + iscSII/pPRONde | 0.6 |
| lipA/pTRC99A + csd/pPRONde | 2600 |
| lipA/pTRC99A + (iscSI + iscU)/pPRONde | 3900 |

*(μg ALA per g dry cell weight)

### EXAMPLE 3

### Analysis of ALA Production by HPLC with Electrochemical Detection

ALA and its dihydro derivative (dihydrolipoic acid or DHLA) can be readily interconverted by applying an electric potential and, therefore, are suitable for measurement by an electrochemical detector. The amount of ALA in reconstituted extracts of the engineered DH10B cells containing one or more constructs are determined by separation on HPLC and monitoring the elution products with an electrochemical detector. Reconstituted extracts of the engineered DH10B cells are prepared as described in EXAMPLE 2, except that methylene chloride extracts are dried under vacuum and reconstituted in the HPLC mobile phase.

Chromatographic separations are carried out using an Ultrasphere C18 column (25 cm×4.6 mm, 5 Tm, Beckman, USA) (or equivalent) with a 30 mm precolumn containing the same packing material. The mobile phase is acetonitrile (approximately 30 to 50%) in a 0.05 M potassium phosphate buffer, pH 2 to 2.5. The electrochemical detection is an Ag—AgCl detector, a Hg—Au electrode system or a glassy carbon electrode system. The detection limit for ALA is in the ng range.

### EXAMPLE 4

### Lipoic Acid Synthesis in S. cerevisiae

To demonstrate that ALA can be produced in other microorganisms, S. cerevisiae lip5 (coding for lipoic acid synthase) and nfs1 (coding for a iscS-like protein) genes were used to express ALA. One skilled in the art will understand that similar methods can be used to produce ALA in any organism or cell desired.

### Construction of Plasmids

Recombinant DNA techniques were carried out according to established procedures (Sambrook et al., Molecular Cloning. A Laboratory Manual, Cold Spring Harbor Laboratory Press, New York, 1989). The sequences of lip5 and nfs1 were obtained from the Stanford yeast genome database. The lip5 and nfs1 genes were prepared by PCR amplification of S. cerevisiae genomic DNA (obtained from ResGen Invitrogen, Corp., Huntsville, Ala.). To PCR amplify lip5 with an Apa I site (forward) and with an XhoI site (reverse), the following primers were used: forward 5′-GGCTAGGGCCCAATAATGTATAGACGATCTG-3′ (SEQ ID NO: 10) and reverse for lip5: 5′-CGGCCTCGAGCATTATTTCATGTTTCTT-3′ (SEQ ID NO: 11). To PCR amplify nfs1 with SpeI site (forward) and a PacI site (reverse) the following primers were used: forward 5′-CCGGACTAGTAAGATGTTGAAATCAACTGCTAC-3′ (SEQ ID NO: 12) and reverse 5′-GCGGCTTAATTAATCAATGACCTGACCATTTGATG-3′ (SEQ ID NO: 13). The restriction sites are underlined. Primers were purchased from Integrated DNA Technologies, Inc.

PCR was performed using the Expand High Fidelity PCR system under the conditions recommended by the manufacturer (Roche Diagnostics Corp., Indianapolis, Ind.); optimal conditions were found to be 50° C. with 2 mM MgCl₂. Each reaction contained the following: 1 μL S. cerevisiae gDNA (10 μg/μL), 0.5 μL of each PCR primer (100 μM), 1 μL of dNTP mixture (10 mM each), 5 μL of “Expand” buffer (with MgCl₂), 1 μL of Expand DNA polymerase, and 41 mL of H₂O. PCR conditions were as follows: 96° C. for 5 minutes; followed by 10 cycles of 96° C. for 30 seconds, 50° C. for 60 seconds, and 72° C. for 105 seconds; then 15 cycles of 96° C. for 30 seconds, 50° C. for 60 seconds, and 72° C. for 105 seconds increasing 5 seconds per cycle; then 10 cycles of 96° C. for 30 seconds, 50° C. for 60 seconds, and 72° C. for 165 seconds; then 72° C. for 7 minutes.

The PCR products were purified from a 1% agarose gel using the Qiagen gel purification kit and eluted from the spin column with 50 μL of EB buffer (10 mM Tris-HCl, pH 8.5). The purified lip5 PCR product (3 μg) was digested with ApaI and Xho1 simultaneously in 100 μL using New England Biolab enzymes and the recommended buffer at 25° C. while the purified nfs1 PCR product (3 μg) was digested with SpeI and PacI simultaneously in 100 μL with the recommended buffer at 37° C. After a 2 hour incubation, the proteins were removed from the digested DNA using a Qiagen PCR clean-up kit. The pESC vector was also digested and purified in a similar manner.

The lip5 PCR product was cloned into the ApaI/XhoI sites of the pESCleu vector (3′ of the Gal 1 promoter, Stratagene, Inc., La Jolla, Calif.) to produce lip5pESCleu. The nfs1 PCR product was cloned into the PacI/SpeI sites of the pESCleu vector (behind the Gal10 promoter) or into the PacI/SpeI sites of lip5pESC. The ligations were carried out using the Rapid DNA ligation kit (Roche Diagnostics Corp., Indianapolis, Ind.) at room temperature (RT) for 20 minutes using a vector to insert molar ratio of 1:5. The ligation reaction mixtures were desalted using a Qiagen PCR clean-up kit and eluted with 50 μL of EB buffer.

### Transformations

E. coli DH10B ElectroMAX cells (Invitrogen Life Technologies, Inc., Carlsbad, Calif.) were used for the first round of transformations. The transformations were carried out by electroporation of 25 μL of cells according to the manufacturer's recommendations (Bio-Rad Gene Pulser II system) with 0.2 cm micro-electroporation cuvettes and the following settings: voltage: 2.5 kV, capacitance: 25 μFarads (microFarads), and impedence: 200 ohms. The DNA/cell mixture was mixed with 0.9 mL SOC medium after electroporation, incubated at 37° C. for 1 hour, and then plated on LB including ampicillin (100 μg/mL). The plates were incubated overnight at 37° C. Plasmid DNA was isolated from liquid cultures (5 mL 2×YT+ampicillin (100 μg/mL) grown overnight at 37° C.) of six colonies picked from the LB+ampicillin plates. A Qiagen mini-prep kit was used for the plasmid purification and the DNA was eluted from the spin columns using EB buffer (50 μL). The purified plasmids were screened by simultaneous digestion with ApaI and XhoI and/or SpeI and PacI as described above. The insert sequences were verified by dideoxynucleotide chain-termination DNA sequencing and one isolate from each construct was chosen for transformation into S. cerevisiae strain YPH500 (Stratagene, Inc., La Jolla, Calif.).

S. cerevisiae strain YPH500 competent cells were prepared using an S.c. EasyComp™ Transformation Kit (Invitrogen Corp., Carlsbad, Calif.). Aliquots (50 μL) were frozen at −80° C. and thawed just prior to use. Transformations were carried out using an S.c. EasyComp™ Transformation Kit following the manufacturer's instructions. For each transformation, 1 μg of vector DNA (lip5pESCleu, nfs1pESCleu, lip5/nfs1pESCleu, or pESCleu) was mixed with 50 μL of competent cells at RT. Solution III (500 μL) from the S.c. EasyComp™ Transformation Kit was added to the DNA/cell mixture and mixed by vortexing vigorously. The transformation mixtures were incubated for 1 hour at 30° C., mixing vigorously every 15 minutes. A 100 μL aliquot from each transformation reaction was spread on SC-leu plates. The plates were incubated for 2 days at 30° C. Four colonies from each plate were picked and analyzed by PCR. One isolate from each construct that generated the expected PCR products (evaluated by agarose gel electrophoresis) was chosen for further study.

Expression experiments with the pESC constructs were performed as described by the manufacturer (Strategene catalog #217451-217455). Specifically, S. cerevisiae str. YPH500 cells carrying one of the described vector constructs or vector alone were grown in 20 mL SC-leu medium containing glucose as the carbon source at 30° C. overnight. SC-leu yeast minimal defined medium (Stratagene) includes the following (per liter): 6.7 g yeast nitrogen base without amino acids (Difco); 0.1 g each adenine, arginine, cysteine, lysine, threonine, tryptophan, and uracil; 0.05 g each aspartic acid, histidine, isoleucine, methionine, phenylalanine, proline, serine, tyrosine, and valine. Leucine is omitted from the medium. This mixture was autoclaved for 15 minutes at 121° C. After cooling, 10% carbon source solution (filter sterilized) was added to bring volume to 1 liter (for solid medium 2% agar added).

The absorbance at 650 nm of each overnight culture was determined and the amount of overnight culture necessary to obtain an absorbance at 650 nm of 0.2 to 0.4 in 150 mL of SC-leu containing 0.2% galactose (induction medium) was calculated. The calculated volume of cells was centrifuged at 1500×g for 10 minutes at 4° C. to pellet the cells and the pellet was resuspended in 2 mL of induction medium and added to the flask containing 150 mL of induction medium. Each construct was grown at 30° C. with shaking at 225 rpm for 17 hours. To harvest, the cell suspensions were divided into two aliquots of 20 and 120 ml.

The smaller aliquot was used for determination of dry cell weight. After centrifugation at 3,000×g for 20 minutes (4° C.), the pellets were re-suspended in 1 mL H₂O, transferred to dried and pre-weighed tubes, and pelleted again by centrifugation at 10,000×g for 3 minutes. The H₂O wash was repeated once and the twice-washed pellets were dried to constant weight at 95° C. The 120 mL aliquots of cells were harvested by centrifugation and washed twice with 5 mL of 25 mM potassium phosphate buffer (pH 7.0). The washed pellets were re-suspended in 1 mL of 25 mM potassium phosphate buffer (pH 7.0) and sealed in 10 mL vials with rubber stoppers. The vials were made anaerobic by flushing with nitrogen for 30 seconds and then evacuating at least 5 times. After the final evacuation, 1 mL of concentrated HCl was added to each vial by injection. The acid mixtures were mixed well and incubated for 4 hours at 110° C. After cooling, the vials were opened and the acidic cell suspensions were extracted four times with 1.0 mL methylene chloride. The combined methylene chloride layers were dried under vacuum and the residues were dissolved in 25 mM potassium phosphate, pH 7.0 (3.0 mL).

Before assaying the re-dissolved residues and cell medium supernatants (saved from the first centrifugation of the cell cultures) were sterilized by filtration through 0.22 μm filters.

### Assay for ALA Production

To determine the amount of ALA produced, the methods described in EXAMPLE 2 using the E. coli lipA::CAT strain described in EXAMPLE 5 were used, except that a sufficient sterile water were added to each of a series of capped tubes (10×750 cm) for a total of 2.0 ml (instead of 1.8 ml) per tube.

As shown in Tables 2 and 3, lipoate synthase and/or Nfs1 resulted in the production of ALA, both intracellularly and extracellularly (secreted). Furthermore, the total amount of ALA secreted into the medium is about ten-fold higher than the amount found intracellularly. Therefore, if desired, one could isolate, purify, and/or concentrate the ALA secreted into the medium, instead of extracting it from the microorganism.

In summary, lipoate synthase and/or Nfs1 resulted in the production of approximately 1 μg ALA per g dry cell weight of intracellular ALA, and about 8.7-11.8 μg ALA per g dry cell weight of extracellular ALA. These results demonstrate that engineered microorganisms can be used to produce levels of ALA which exceed endogenous levels (<0.5 μg ALA per g dry cell weight intracellular; <7 μg ALA per g dry cell weight extracellular, in eukaryotes), for example at least 200 μg ALA per g dry cell weight in prokaryotes or 1 μg ALA per g dry cell weight in eukaryotes.

TABLE 2
Intracellular ALA content detected in S. cerevisiae

| Construct | ALA content* |
|---|---|
| pESCleu/YPH500 | 0.3 |
| lip5pESCleu/YPH500 | 0.7 |
| nfs1pESCleu/YPH500 | 1.0 |
| (lip5 + nfs1)pESCleu/YPH500 | 1.1 |

*μg ALA per g dry cell weight

TABLE 3
Extracellular ALA content from S. cerevisiae

| Construct | ALA content* |
|---|---|
| pESCleu/YPH500 | 7.1 |
| lip5pESCleu/YPH500 | 12.1 |
| nfs1pESCleu/YPH500 | 11.3 |
| (lip5 + nfs1)pESCleu/YPH500 | 8.7 |

*μg ALA secreted per g dry cell weight

### EXAMPLE 5

### Construction and Use of E. coli lipA::CAT

To develop an alternative method for assaying for ALA production, an E. coli strain with the lipA (lipoic acid synthase) gene disrupted by the CAT (chloramphenicol acetyltransferase) gene was generated by the method of Datsenko and Wanner (Proc. Natl. Acad. Sci. USA 97: 6640-5, 2000). As a result of the disruption of the lipA gene, this E. coli strain is dependent on external sources of ALA for its growth.

E. coli strains BW 25113 carrying pKD46 and BW 25141 carrying pKD3 were obtained from the E. coli Genetic Stock Center, Yale University, New Haven, Conn. The CAT gene on pKD3 was amplified by PCR using SEQ ID NO: 18 (AAC GCT TTC CTT CGT AAT TCG CAA CTG GAA CAC GCA CGCT GT GTA GGC TGG AGC TGC TTC) and SEQ ID NO: 19 (CGG GTT TTT TAT CAG ACA GAT GTA AGT AAT TAT TAC AGG A CA TAT GAA TAT CCT CCT TAG), each of which is composed of 40 nucleotides at the 5′ end that are complementary to regions 5′ or 3′ of the E. coli lipA gene, respectively, and 20 nucleotides at the 3′ end complementary to the CAT gene. PCR was performed with Taq polymerase using 30 cycles of 95° for 30 seconds, 45° for 30 seconds, 72° C. for 1 minute; followed by 72° C. for 7 minutes. The PCR product was recovered with QIAquick PCR Purification Kit and concentrated by ethanol precipitation. The recombinogenic functions carried on pKD46 in BW25113 were induced with 1 mM arabinose as described by Datsenko and Wanner, and electrocompetent cells prepared by washing twice with ⅓ volume 10% glycerol, followed by resuspension in 0.005 volumes of 10% glycerol.

One μl of purified and concentrated PCR product was electroporated into the induced electrocompetent BW25113/pKD46 and transformants were plated on LB medium containing 25 μg/ml chloramphenicol. Transformants resistant to chloramphenicol were picked and passaged on LB without antibiotic at 43° C. to cure the plasmid, and re-plated for single colonies on LB containing 25 μg/ml chloramphenicol, 0.1% glucose, and 5 ng/ml ALA. Integration of the CAT gene into the lipA locus in chloramphenicol-resistant colonies was confirmed by colony PCR using SEQ ID NO: 20 (GGT ATC TAT GGG TGA GAT TAG T) and SEQ ID NO: 21 (GTC CTT AAA TGA GGA GCA AAT AGA T) which are located outside of the lipA region. PCR products from undisrupted lipA and lipA::CAT colonies were distinguished on the basis of size (1590 vs. 1641 bp) and the presence of a PvuII restriction endonuclease site in the CAT gene; digestion of the lipA::CAT PCR product by. PvuII yields fragments of 941 and 700 bp.

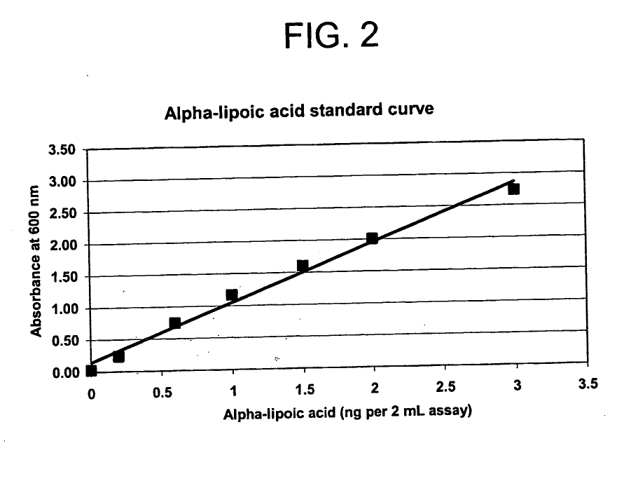

The lipA::CAT strain was used to assay for ALA by the method of EXAMPLE 2. A standard curve with. 0 to 3.0 ng ALA generated the response shown in FIG. 2 and confirms linear dependence of growth of the lipA::CAT strain on ALA. This result demonstrates that the lipA::CAT strain can be used to assay for ALA production.

### EXAMPLE 6

### Recombinant Expression

With publicly available lipoate synthase, IscS, IscU, and Nfs1 cDNA and amino acid sequences, as well as variants, homologs, polymorphisms, mutants, fragments and fusions thereof, the expression and purification of any protein, such as an enzyme, by standard laboratory techniques is enabled. One skilled in the art will understand that proteins disclosed herein and variants thereof can be produced recombinantly in any cell or organism of interest.

Methods for producing recombinant proteins are well known in the art. Therefore, the scope of this disclosure includes recombinant expression of any protein or fragment thereof, such as an enzyme. For example, see U.S. Pat. No.: 5,342,764 to Johnson et al.; U.S. Pat. No.: 5,846,819 to Pausch et al.; U.S. Pat. No.: 5,876,969 to Fleer et al. and Sambrook et al. (Molecular Cloning: A Laboratory Manual, Cold Spring Harbor, N.Y., 1989, Ch. 17).

Briefly, partial, full-length, or variant cDNA sequences, which encode for a protein or peptide, can be ligated into an expression vector, such as a bacterial expression vector. Proteins and/or peptides can be produced by placing a promoter upstream of the cDNA sequence. Examples of promoters include, but are not limited to lac, trp, tac, trc, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

Vectors suitable for the production of intact native proteins include pKC30 (Shimatake and Rosenberg, 1981, Nature 292:128), pKK177-3 (Amann and Brosius, 1985, Gene 40:183) and pET-3 (Studiar and Moffatt, 1986, J. Mol. Biol. 189:113). A DNA sequence can be transferred to other cloning vehicles, such as other plasmids, bacteriophages, cosmids, animal viruses and yeast artificial chromosomes (YACs) (Burke et al., 1987, Science 236:806-12). These vectors can be introduced into a variety of hosts including somatic cells, and simple or complex organisms, such as bacteria, yeast, fungi (Timberlake and Marshall, 1989, Science 244:1313-7), invertebrates, plants (Gasser and Fraley, 1989, Science 244:1293), and mammals (Pursel et al., Science 244:1281-8, 1989), which are rendered transgenic by the introduction of the heterologous cDNA.

For expression in mammalian cells, a cDNA sequence can be ligated to heterologous promoters, such as the simian virus SV40, promoter in the pSV2 vector (Mulligan and Berg, 1981, Proc. Natl, Acad. Sci. USA 78:2072-6), and introduced into cells, such as monkey COS-1 cells (Gluzman, 1981, Cell 23:175-82), to achieve transient or long-term expression. The stable integration of the chimeric gene construct may be maintained in mammalian cells by biochemical selection, such as neomycin (Southern and Berg, 1982, J. Mol. Appl. Genet. 1:327-41) and mycophoenolic acid (Mulligan and Berg, 1981, Proc. Natl. Acad Sci. USA 78:2072-6).

The transfer of DNA into eukaryotic cells is a conventional technique. The vectors are introduced into the recipient cells as pure DNA (transfection) by, for example, precipitation with calcium phosphate (Graham and vander Eb, 1973, Virology 52:466) strontium phosphate (Brash et al., 1987, Mol. Cell Biol. 7:2013), electroporation (Neumann et al., 1982, EMBO J. 1:841), lipofection (Feigner et al., 1987, Proc. Natl. Acad. Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Natl. Acad. Sci. USA 77:2163-7), or pellet guns (Klein et al., 1987, Nature 327:70). Alternatively, the cDNA can be introduced by infection with virus vectors, for example retroviruses (Bernstein et al., 1985, Gen. Engrg. 7:235) such as adenoviruses (Ahmad et al., 1986, J. Virol. 57:267) or Herpes (Spaete et al., 1982, Cell 30:295).

### EXAMPLE 7

### Overexpression of Iron Sulfur Cluster Genes

In addition to the introduction of exogenous genes on plasmid vectors, overexpression of the iscS and iscU genes can be achieved by other means, for example by decreasing or eliminating the repression of expression of these genes. For example, in E. coli, the regulatory ORF encoded by the iscR gene can be functionally deleted to decrease or eliminate repression of expression of iscS and iscU genes (Schwartz et al., Proc. Natl. Acad. Sci. USA 98: 14895-900, 2001). The iscR gene can be disrupted by insertional inactivation using the method of Datsenko and Wanner (Proc. Natl. Acad Sci. USA 97: 6640-5, 2000) as described in EXAMPLE 5, followed by the excision of the insertion using the FLP function encoded on plasmid pCP20 as described by Datsenko and Wanner. The resultant strain (ΔiscR camS) expresses the isc cluster genes, including iscS and iscU, constitutively. Introduction of the exogenous lipA gene, such as on plasmid lipA/pTRC99A, would result in overproduction of the lipoate synthase enzyme in the presence of overexpressed polypeptides that assemble or regenerate Fe—S clusters, and hence in the supraendogenous biosynthesis of ALA.

### EXAMPLE 8

### Uses of ALA

ALA produced using the engineered microorganisms and methods disclosed herein, can be used for any application in which ALA is desired. Currently, ALA is produced using chemical synthesis, which results in a racemic mixture of S- and R-ALA. The present disclosure provides methods and organisms which permit the production of large amounts of the non-racemic form, R-ALA, which can be used to produce nutraceuticals and pharmaceuticals.

ALA can be used to treat or reduce or alleviate the symptoms of many diseases or disorders. For example, ALA can be used reduce the symptoms of diabetes (types I and II), such as decreasing diabetic polyneuropathy (caused by diabetes, renal failure, tumors); glycation of proteins, cataract formation, and cell destruction leading to type I diabetes; and enhancing glucose uptake in type II (non-insulin dependent). ALA can also be used to reduce the symptoms of glaucoma. Other uses include, but are not limited to: decreasing the replication of HIV; decreasing the effects of mushroom, heavy metal and radiation poisoning; decreasing the symptoms associated with Lyme disease, Alzheimer's disease (increase cognitive function), and chronic/toxic liver disorders. Furthermore, ALA can be used in topical applications, for example to the skin as an antioxidant (by reducing skin inflammation induced by UVB radiation and chemical irritants (anti-aging properties). In addition, ALA can be used as a nutraceutical (food additive).

The ALA produced using the engineered microorganisms and methods disclosed herein, can be administered to a subject alone, or in combination with a carrier (such as a pharmaceutical carrier) and/or other compositions.

In view of the many possible embodiments to which the principles of our disclosure may be applied, it should be recognized that the illustrated embodiments are only particular examples of the disclosure and should not be taken as a limitation on the scope of the disclosure. Rather, the scope of the disclosure is in accord with the following claims. We therefore claim as our invention all that comes within the scope and spirit of these claims.

1. An engineered microorganism, wherein the microorganism produces at least 200 μg α-lipoic acid (ALA) per g dry cell weight of the engineered microorganism.

2. The engineered microorganism of claim 1, wherein the microorganism comprises an exogenous nucleic acid encoding a lipoic acid synthase polypeptide.

3. The engineered microorganism of claim 2, wherein the microorganism further comprises an exogenous nucleic acid encoding at least one polypeptide that assembles or regenerates an Fe—S cluster of the lipoic acid synthase polypeptide.

4. The engineered microorganism of claim 3, wherein the at least one polypeptide that assembles or regenerates an Fe—S cluster of the lipoic acid synthase polypeptide is an iron-sulfur cluster S (IscS) polypeptide.

5. The engineered microorganism of claim 4, wherein the IscS polypeptide is cysteine sulfinic desulfinase (Csd).

6. The engineered microorganism of claim 3, wherein the at least one polypeptide that assembles or regenerates an Fe—S cluster of the lipoic acid synthase polypeptide is an iron-sulfur cluster U (IscU) polypeptide.

7. The engineered microorganism of claim 3, wherein the at least one polypeptide that assembles or regenerates an Fe—S cluster of the lipoic acid synthase polypeptide is an IscS polypeptide and an IscU polypeptide.

8. The engineered microorganism of claim 1, wherein the microorganism comprises an exogenous lipoic acid synthase polypeptide, an exogenous IscS polypeptide, and an exogenous IscU polypeptide.

9. The engineered microorganism of claim 1, wherein the microorganism is a prokaryote.

10. The engineered microorganism of claim 9, wherein the prokaryote is of the Escherichia genus.

11. The engineered microorganism of claim 10, wherein the prokaryote is Escherichia coli.

12. The engineered microorganism of claim 2, wherein the microorganism is lacking an endogenous transcriptional regulator ORF.

13. The engineered microorganism of claim 12, transcriptional regulator ORF is as IscR.

14. A method of making ALA, comprising extracting ALA from the engineered microorganism of claim 1.

15. The method of claim 14, wherein the engineered microorganism comprises an exogenous lipoic acid synthase polypeptide, an IscS polypeptide, or an exogenous IscU polypeptide.

16. The method of claim 14, wherein the engineered microorganism comprises an exogenous lipoic acid synthase polypeptide, an IscS polypeptide, and an exogenous IscU polypeptide.

17. A composition comprising ALA produced by the engineered microorganism of claim 1.

18. A nutraceutical comprising ALA produced by the engineered microorganism of claim 1.

19. A pharmaceutical comprising ALA produced by the engineered microorganism of claim 1.

20. An isolated nucleic acid comprising a first expression control element operably linked to an nucleic acid encoding a lipoic acid synthase polypeptide and a second expression control element operably linked to a nucleic acid encoding an IscS polypeptide and/or an IscU polypeptide.

21. The isolated nucleic acid of claim 20, wherein the IscS polypeptide is Csd.

22. A vector comprising the isolated nucleic acid of claim 20.

23. A cell comprising the isolated nucleic acid of claim 20.

24. An engineered microorganism, wherein the microorganism secretes at least a 2-fold greater amount of ALA than an amount of ALA found intracellularly.

25. The engineered microorganism of claim 24, wherein the microorganism secretes at least a 5-fold greater amount of ALA than an amount of ALA found intracellularly.

26. The engineered microorganism of claim 24, wherein the microorganism secretes at least a 10-fold greater amount of ALA than an amount of ALA found intracellularly.

27. The engineered microorganism of claim 24, wherein the microorganism secretes at least 8.7 μg ALA per g dry cell weight and contains less than 1.5 μg ALA per g dry cell weight intracellularly.

28. The engineered microorganism of claim 24, wherein the microorganism is a eukaryote.

29. The engineered microorganism of claim 28, wherein the eukaryote is a member of the genus Saccharomyces.

30. The engineered microorganism of claim 29, wherein the eukaryote is S. cerevisiae.

31. The engineered microorganism of claim 24, wherein the microorganism comprises an exogenous nucleic acid encoding a lipoate synthase polypeptide.

32. The engineered microorganism of claim 31, wherein the microorganism further comprises an exogenous nucleic acid encoding at least one polypeptide that assembles or regenerates an Fe—S cluster of the lipoate synthase polypeptide.

33. The engineered microorganism of claim 32, wherein the at least one polypeptide that assembles or regenerates an Fe—S cluster of the lipoate synthase polypeptide is an Nfs1 polypeptide.

34. The engineered microorganism of claim 24, wherein the microorganism comprises an exogenous lipoate synthase polypeptide and an exogenous Nfs1 polypeptide.

35. A nucleic acid construct comprising a first expression control element operably linked to a nucleic acid encoding a lipoate synthase polypeptide and a second expression control element operably linked to a nucleic acid encoding an Nfs1 polypeptide.

36. A method of making ALA, comprising purifying ALA secreted by the engineered microorganism of claim 24.

37. A composition comprising ALA produced by the engineered microorganism of claim 24.

38. A nutraceutical comprising ALA produced by the engineered microorganism of claim 24.

39. A pharmaceutical comprising ALA produced by the engineered microorganism of claim 24.

40. A method of producing ALA comprising culturing an engineered microorganism expressing an exogenous nucleic acid encoding a lipoic acid synthase polypeptide under conditions which permit the microorganism to produce ALA.

41. The method of claim 40, further comprising expressing an exogenous nucleic acid encoding at least one polypeptide that assembles or regenerates an Fe—S cluster of the lipoic acid synthase polypeptide.

42. The method of claim 41, wherein the at least one polypeptide that assembles or regenerates an Fe—S cluster of the lipoic acid synthase polypeptide is an IscS polypeptide and/or an IscU polypeptide.

43. The method of claim 41, wherein the at least one polypeptide that assembles or regenerates an Fe—S cluster of the lipoic acid synthase polypeptide is an Nfs1 polypeptide.

44. The method of claim 40, wherein the microorganism produces at least 200 μg ALA per g dry cell weight.

45. The method of claim 40, wherein the microorganism secretes at least a 2-fold greater amount of ALA than an amount of ALA found intracellularly.
